(19) United States
(12) Patent Application Publication
(10) Pub. No.: US 2023/0190655 A1
(43) Pub. Date: Jun. 22, 2023

(54) METHODS FOR CONSOLIDATING ANTIBIOTIC-ELUTING POLYMERIC MATERIALS

(71) Applicant: THE GENERAL HOSPITAL CORPORATION, Boston, MA (US)

(72) Inventors: Orhun K. MURATOGLU, Cambridge, MA (US); Ebru ORAL, Newton, MA (US); Ali Eren ATICI, Melrose, MA (US)

(21) Appl. No.: 17/291,588

(22) PCT Filed: Nov. 12, 2019
(86) PCT No.: PCT/US2019/060904

§ 371 Date: May 5, 2021

Related U.S. Application Data
(60) Provisional application No. 62/760,405, filed on Nov. 13, 2018.

(51) Int. Cl. A61K 9/16; A61K 31/7036; A61J 3/00; B29B 9/16; B29B 9/10

(52) U.S. Cl. CPC A61K 9/1641; A61K 31/7036; A61K 9/1682; A61J 3/00; B29B 9/16; B29B 9/10; B29K 2105/0011

(57) ABSTRACT

Methods of making consolidated blend(s) of polymeric material(s) with one or more therapeutic agents (such as an antibiotic) are provided, wherein the method comprises the steps of providing a polymeric material, blending the polymeric material with one or more therapeutic agent(s), pelletizing the blended polymeric material, environmentally treating by various approaches the pelletized polymeric material, and consolidating the environmentally treated pellet. Products made by the methods and uses of the products also are provided.

### FIELD OF THE INVENTION

The present invention relates to methods for making polymeric materials using pelletization and treatment in various environments. It also relates to methods of incorporating therapeutic agents in polymeric materials. It also relates to methods of making medical devices comprised of polymeric materials incorporated with therapeutic agents.

### BACKGROUND OF THE INVENTION

Total joint arthroplasty, in the hip, knee, shoulder, and other joints, produces very successful outcomes. One of the major reasons for revision is the infection of the reconstructed joint. The implants used during surgery are metallic, ceramic, or polymeric in nature and are prone to colonizing bacteria. One way to reduce the rate of infection is to improve the surfaces of the implants used to reduce the colonization of bacteria. For example, antibiotic coatedMoaded materials can be used to inhibit bacterial adhesion and colonization. Antibiotic loaded polymethyl methacrylate (PMMA) bone cement has been in clinical use in total joint arthroplasty surgery to prophylactically reduce infections. Antibiotic loaded bone cement has been somewhat successful in reducing the infection rate. However, once infected, the implants have to be removed, the joint be debrided, and in some cases temporary articulating or static spacer implants are implanted. These spacer implants are manufactured from PMMA bone cement and contain various antibiotics. Temporal release of antibiotics to the surgical site helps clear the infection (Stevens C M, Tetsworth K D, Calhoun J H, Mader J T. An articulated antibiotic spacer used for infected total knee arthroplasty: a comparative in vitro elution study of Simplex and Palacos bone cements. J Orthop Res Off Publ Orthop Res Soc. 2005; 23(1):27-33.). The spacer implants are temporary in nature and they are typically replaced within six months of implantation with permanent implants. However, PMMAs present several disadvantages such as the occurrence of chemical necrosis caused by non-polymerized monomer residues, and low toughness (Belt, H. V. D., Neut, D., Schenk, W., Horn, J. R. V., Mei, H. C. V. D., & Busscher, H. J. (2001). Infection of orthopedic implants and the use of antibiotic-loaded bone cements: a review. Acta Orthopaedica Scandinavica, 72(6), 557-571.). Patients are largely immobilized during treatment due to PMMA spacers not being able to bear the full weight of the patients.

According to the invention, therapeutic agents, such as antibiotics, can be incorporated into ultra-high molecular weight polyethylene (UHMWPE) implants typically used in total joint arthroplasty for local delivery of these therapeutic agents. UHMWPE is a better candidate than PMMA bone cement as an articulating spacer and a delivery device eluting antibiotics because of its superior mechanical strength and markedly improved wear resistance in comparison to bone cement.

Typically, acetabular liners in total hips, tibial inserts in total knees, glenoid components in total shoulders are fabricated from UHMWPE. Prophylaxis, to reduce acute and chronic infections, can also be carried out by using therapeutic agent containing UHMWPE implants not only in revision surgery but also in primary surgery.

UHMWPE is typically sourced in powder form. The UHMWPE powder can be blended with therapeutic agent powder or therapeutic agent liquid or therapeutic agent solution. Subsequently the blend can be consolidated either into a final implant shape or into a form that can be machined into implant shape. The consolidation step utilizes elevated temperature and pressure to fuse the UHMWPE powder together. Therefore, consolidation can induce thermal degradation of the therapeutic agent. According to the invention, it can be desirable with some hygroscopic therapeutic agents to subject the therapeutic agents to a dehydration step before consolidation together with UHMWPE to minimize the thermal degradation. One such therapeutic agent that is commonly used is gentamicin sulfate and another one is tobramycin or vancomycin hydrochloride. In addition to the dehydration step, it is also desirable to use low oxygen environments during consolidation to minimize potential oxidation of the therapeutic agent. Methods for consolidating blends of therapeutic agents and polymeric material, for example UHMWPE, while minimizing oxidative and thermal changes to the incorporated antibiotic(s) are described.

Teachings of production materials, approaches, methodologies and conditions are set forth in attorney docket number MGH 23975, titled Implant Surfaces for Pain Control, U.S. Provisional Patent Application No. 62/330,478 filed 2 May 2016 (now PCT/US2017/029789, filed 27 Apr. 2017); and attorney docket number MGH 23709, titled Drug Eluting Polymer Composed of Biodegradable Polymers Applied to Surface of Medical Device, U.S. Provisional Patent Application No. 62/291,856, filed 5 Feb. 2016 (now PCT/US2017/016506, filed 3 Feb. 2017). These production materials, approaches, methodologies and conditions are incorporated herein by reference.

### BRIEF DESCRIPTION OF THE DRAWINGS

FIG. 1 shows chemical structures of Gentamicin isomers.

FIG. 2 shows images of discoloration patterns of 4.22 wt. % GS/UHMWPE block that were prepared as described in Example 6.

FIG. 3 shows non-discolored, blended, pelletized, vacuum-treated and compression molded 4.22 wt. % GS/UHMWPE block.

FIG. 4 5, 6 shows XPS spectra of discolored 4.22 wt. % GS/UHMWPE blocks that were prepared as described in Example 6.

FIG. 7 8, 9 shows XPS spectra of non-discolored 4.22 wt. % GS/UHMWPE blocks that were prepared as described in Example 6.

FIG. 10 shows image of block of virgin UHMWPE with GS/UHMWPE layer on top as described in Example 8.

FIG. 11 shows calibration curve of GS concentration vs isomer peaks' intensity, and representative total ion chromatogram of GS obtained from LC/MS and mass spectrum of GS in PBS.

FIG. 12 shows incremental GS elution profiles in μg/ml of 4.22 wt. % and 8 wt. % GS containing UHMWPE samples.

FIG. 13 shows incremental GS elution per surface area for 4.22 wt. % and 8 wt. % GS containing UHMWPE samples.

FIG. 14 shows effect of manufacturing technique on elution for 4.22 wt. % and 8 wt. % GS containing UHMWPE samples.

FIG. 15 shows design of mold plunger assemblies with thermocouple holes.

FIG. 16 shows heating performance comparison of stainless steel and aluminum bronze mold plunger assemblies.

FIG. 17 shows cooling performance comparison with stainless steel and aluminum bronze mold plunger assemblies.

FIG. 18 shows discoloration of 5 min. microwave-treated, compression molded GS/UHMWPE blended with 4.22% wt. GS.

FIG. 19 shows 10 min. microwave-treated, compression molded GS/UHMWPE blended with 4.22% wt. GS.

### SUMMARY OF THE INVENTION

In one embodiment, the invention provides methods of making a consolidated polymeric material blend with additive(s) comprising: providing a polymeric material, blending the polymeric material with an additive(s), pelletizing the polymeric material blend, dehydrating the pelletized polymeric material blend, and consolidating the dehydrated blend.

In one embodiment of the invention, there are provided methods of making a medical implant comprising: providing a polymeric material, blending the polymeric material with an additive(s), pelletizing the polymeric material blend, dehydrating the pelletized polymeric material blend, and consolidating the dehydrated blend. The consolidated polymeric material can be machined into an implant shape.

One embodiment provides methods of making a consolidated blend of polymeric material with an additive(s) comprising: providing a polymeric material, blending the polymeric material with an additive(s), dehydrating the polymeric material blend, and consolidating the dehydrated blend.

Another embodiment provides methods of making a medical implant comprising: providing a polymeric material, blending the polymeric material with an additive(s), dehydrating the polymeric material blend, and consolidating the dehydrated blend. The consolidated polymeric material can be machined into an implant shape.

In any of the embodiments, the dehydrated polymeric material blend can be pelletized before consolidation. The polymeric material, the additive(s), and/or the polymeric material blend are subjected to dehydration at any step of the fabrication. The dehydration can include several cycles of dehydration and different types of dehydration such as vacuum dehydration, heat dehydration under vacuum, microwave dehydration, microwave dehydration under vacuum.

One embodiment provides methods of making Gentamicin Sulfate/UHMWPE (GS/UHMWPE) medical implant comprising: providing UHMWPE, blending the UHMWPE with GS to obtain a GS/UHMPWE blend, pelletizing the GS/UHMWPE blend, thereby forming a GS/UHMWPE pellet, dehydrating the GS/UHMWPE pellet, consolidating the environmentally treated GS/UHMWPE pellet.

Another embodiment provides methods of making consolidated Gentamicin Sulfate/UHMWPE (GS/UHMWPE) medical implant comprising: providing UHMWPE, blending the UHMWPE with GS to obtain a GS/UHMPWE blend, pelletizing the GS/UHMWPE blend, thereby forming a GS/UHMWPE pellet, dehydrating the GS/UHMWPE pellet, consolidating the environmentally treated GS/UHMWPE pellet. The consolidated GS/UHMWPE can be machined into an implant shape.

The invention also provides medical implants comprising layers of polymeric material. One embodiment provides medical implants made by the method of making layered, consolidated polymeric material containing a therapeutic agent comprising:
a. Providing a first polymeric material;
b. Providing a second polymeric material;
c. Blending one or more of the polymeric materials with one or more therapeutic agents at least one of which is an antibiotic;
d. Partially consolidating/pelletizing one or more of the polymeric materials;
e. Environmentally treating one or more of the polymeric materials;
f. Layering the polymeric materials;
g. Completely consolidating the polymeric materials, thereby forming a medical implant.

One embodiment provides medical implants made by methods of making layered, consolidated, interlocked hybrid material containing a therapeutic agent comprising:
a. Providing a polymeric material;
b. Providing a second metallic material;
c. Blending the polymeric material with one or more therapeutic agents at least one of which is an antibiotic;
d. Layering the blended polymeric material and the second metallic material;
e. Partially consolidating/pelletizing; thereby forming an interlocked hybrid pellet;
f. Environmentally treating the interlocked, hybrid pellet;
g. Completely consolidating the environmentally treated interlocked, hybrid pellet, thereby forming a medical implant.

In any of the embodiments, wherein polymeric materials are layered and consolidated into the shape or close to the shape of a medical implant, some machining may be used afterwards to obtain the final medical implant to be packaged and sterilized.

An embodiment provides medical implants made by methods of making layered, interlocked, hybrid material containing a therapeutic agent comprising:
a. Providing a polymeric material;
b. Blending one or more of the polymeric materials with one or more therapeutic agents at least one of which is an antibiotic;
c. Microwaving the blended polymeric material;
d. Providing a second metallic material;
e. Layering the blended and polymeric material and the second metallic material;
f. Completely consolidating the materials, thereby forming a medical implant.

An embodiment provides medical implants made by the methods of making layered, consolidated, interlocked, hybrid material containing a therapeutic agent comprising:
a. Providing a first polymeric material;
b. Providing a second polymeric material;
c. Blending one or more of the polymeric materials with one or more therapeutic agents at least one of which is an antibiotic;
d. Providing a third metallic material;
e. Layering the first and second polymeric materials and the third metallic material;
f. Partially consolidating/pelletizing the layered materials; thereby forming an interlocked, hybrid pellet;
g. Environmentally treating the interlocked, hybrid pellet;
h. Completely consolidating the environmentally treated interlocked hybrid pellet, thereby forming a medical implant.

One embodiment provides methods of making layered, consolidated polymeric material containing a therapeutic agent comprising:
a. Providing a first polymeric material;
b. Providing a second polymeric material;
c. Blending one or more of the polymeric materials with one or more therapeutic agents at least one of which is an antibiotic;
d. Partially consolidating/pelletizing one or more of the polymeric materials;
e. Environmentally treating one or more of the polymeric materials;
f. Layering the polymeric materials;
g. Completely consolidating the polymeric materials.

In one embodiment the finished implant made form polymeric material blend has rough surfaces to increase the overall surface area. The rough surfaces can be achieved by machining the implants after consolidation in a manner that creates a rough surface. Another method of doing so can be by direct compression molding the implants with molds that have rougher surface finishes. The surface roughness of the implant can have wide ranges with a wide range of Rₐ, R_{q}, Rᵥ, Rₚ, Rₜ, R_{y}, Rₛₖ, Rₖᵤ, Rₜₘ values without any limitations.

In any of the embodiments the consolidated polymeric blend can be machined into an implant shape. The implant can be packaged and sterilized.

In any of the embodiments, at least one of the therapeutic agent(s) in the additive blended with the polymeric material is an antibiotic.

In any of the embodiments, the additive is a therapeutic agent(s) and/or an antioxidant(s) and/or a desiccant(s) and/or a dehydration agent(s), or a mixture thereof. In some embodiments the additive can be a mixture of therapeutic agent(s) and/or antioxidant(s) and/or desiccant(s) and/or dehydration agent(s).

In some embodiments, additive(s) can be dehydrated and/or polymeric material(s) can be dehydrated before any, some, or all of them are blended together to form a polymeric material blend. In other embodiments, additive(s) and/or polymeric material(s) are blended together to form a polymeric material blend, which polymeric material blend can be then dehydrated. The polymeric material blend can be then consolidated either in a dehydrated state or a non-dehydrated state.

In any embodiments, blending of the polymeric material with additive(s) can be done by dry blending and/or by wet blending to obtain a polymeric material blend. Polymeric material can be blended with an antioxidant(s) first, then blended with a therapeutic agent(s) or vice versa to obtain a polymeric material blend. The percentage by weight of therapeutic agent(s) in the polymeric material blend can be from 0.001 wt % to 50 wt % or 1%, 2%, 3%, 4%, 5%, 6%, 7%, 8%, 9%, 10% or more or less than 1% or in between those percentages. The percentage by weight of antioxidant(s) in the polymeric material blend can be from 0.001% to 50 wt. % or 1%, 2%, 3%, 4%, 5%, 6%, 7%, 8%, 9%, 10% or more or less than 1% or in between those percentages. The polymeric material blend could be in powder form or could be consolidated into a solid form. In some embodiments the polymeric material can be blended with therapeutic agent(s) only. In other embodiments polymeric material can be blended with therapeutic agent(s) and/or antioxidant(s) and/or desiccant(s) and/or dehydration agent(s).

In any of the embodiments, pelletization of the blended polymeric material can be done by compression. By pelletization is meant partial consolidation. To pelletize, polymeric material with or without the additive(s) can be placed inside a mold and partially consolidated. Pelletization can be done at ambient pressure by simply heating. It can also be done at elevated pressures, such as 10 MPa, 20 MPa, 40 MPa, less than 10 MPa or more than 40 MPa. Pelletization can be done at room temperature under elevated pressure. Pelletization can be done with or without active pressurization at elevated temperatures such as 25° C., 30° C., 40° C., 50° C., 60° C., 70° C., 80° C., 90° C., 100° C., 110° C., 120° C., 130° C., 145° C., 150° C., 160° C., 170° C., 180° C., 250° C., 300° C. or above or at any temperature in-between. Pelletization can be done by cold pressing, that is, by pressurizing the polymeric material blend filled mold without active heating. Pelletization or partial consolidation can be done at a combination of temperatures and pressures. Pelletization or partial consolidation can be done for less than 1 min to several days. Preferably the duration of pelletization or partial consolidation can be between 5-20 mins, or 20-40 mins.

In some embodiments, consolidation of polymeric material blend can be done by compression molding. The polymeric material blend can be placed inside the mold and pressurized by using a plunger that fits in the mold cavity. Alternatively, polymeric material blend can be consolidated by warm or hot isostatic pressing. Compression molding can be at 10 MPa, 20 MPa, 40 MPa, less than 10 MPa or more than 40 MPa of pressure. Compression molding can be at 0° C., 5° C., 10° C., 20° C., 25° C., 30° C., 40° C., 50° C., 60° C., 70° C., 80° C., 90° C., 100° C., 110° C., 120° C., 130° C., 140° C., 150° C., 160° C., 170° C., 180° C., 190° C., 200° C., 210° C., 220° C., 230° C., 240° C., 250° C., 260° C., 270° C., 280° C., 290° C., 300° C., 310° C., 320° C. or above or at any temperature in-between. Preferably at 130° C.-140° C. More preferably at 140° C.-150° C. Most preferably at 160° C.-180° C. Any of the compression molding can be for less than 1 min to several days. Certain additives used in polymeric material blend can be temperature sensitive. In those cases, the consolidation temperature and duration are optimized to minimize the degradation of the constituents in the polymeric material blend.

In some embodiments, the consolidated polymeric material can be machined into a desired geometry, for example comprising holes, indentations; tapered holes, indentations; blunt holes, indentations; or screw holes. The desired geometry could be a medical implant, such as an acetabular liner, tibial insert, or shoulder glenoid, or like.

In any of the embodiments, polymeric material blend in a geometry, for example the medical device, intended for implantation, can be sterilized; sterilization can be done by a gas method such as with ethylene oxide (EtO) gas. Or it can be sterilized by irradiation such as with electron-beam (e-beam) or gamma radiation.

In some embodiments, the consolidated polymeric material blend can be fabricated through “direct compression molding” (DOM), which is compression molding using parallel plates or any plate/mold geometry which can result in a solid form of the polymeric material in the shape of an implant or implant preform. Preforms are generally oversized versions of implants, where some small amount of machining of the preform can result in the final shape of the implant. In some embodiments certain features of the final implant shape may be machined after direct compression molding. In other embodiments no additional machining is needed to obtain the final implant shape after direct compression molding.

In some embodiments, the polymeric material blend can be lyophilized. In any of the embodiments, the duration of lyophilization can be 10 min, 20 min, 30 min, 40 min, 50 min, 1 h, 2 h, 3 h, 4 h, 5 h, 6 h, 7 h, 8 h, 9 h, 10 h, 15 h, 24 h or less than 10 min or more than 24 h or anywhere in between. The polymeric material blend can be first frozen by cooling down to a temperature of 0° C., −10° C., −20° C., −30° C., −40° C., −50° C., −60° C., −70° C., −80° C., −90° C., −100° C., −150° C., −196° C. or any temperature in between. The frozen polymeric material blend can be then subjected to heat and/or vacuum to sublimate the water molecules. Heating during sublimation can be at 10° C., 20° C., 30° C., 40° C., 50° C. or below 10° C. or above 50° C. or any temperature in between. Vacuum during sublimation can be at around ambient pressure, above ambient pressure or below ambient pressure.

In any of the embodiments, the consolidated polymeric material blend can be further machined to increase its surface area. For instance, the consolidated polymeric material can be machined to introduce different diameter holes (0.1 mm to 5 mm or larger) with different depths (0.1 mm to 5 mm or deeper). The holes can be less than 0.1 mm in diameter and/or less than 0.1 mm in depth. The machined holes can be circular, square, rectangular or any other shape in cross-section and the can cover all surfaces of the consolidated polymeric material blend. In some embodiments, the holes will only cover some of the surfaces. The holes are distributed uniformly or non-uniformly. In any of the embodiments, the surface can be machined to be textured in any random or repeating pattern. For example, structures can be machined such as channels, reservoirs or interconnected depots in any shape or dimension. The consolidated polymeric material blend can first be machined into medical implant shape and then further machined to increase the surface area of the medical implant shape. Increasing the surface area has the benefit of increasing the elution of the additive(s), such as the antibiotics, from the medical implant.

In one embodiment, polymeric material blend can be consolidated using direct compression molding with a specific surface texture on the molded surfaces. The surface texture can be achieved by texturing the surfaces inside the cavity of the mold and/or the surface of the plunger. Texturing helps increase surface area and increase elution of the additive(s).

In any of the embodiments, polymeric material blend(s) can be layered with other polymeric material blend(s) during pelletization or compression molding. In one embodiment, one or more such layer(s) are first pelletized and dehydrated before consolidation. The layers can contain the same therapeutic agent or different therapeutic agents or no therapeutic agents. The layers can also contain the same therapeutic agent at different concentrations. Layers can be any thickness such as 1 mm or 2 mm or 3 mm or 4 mm or 5 mm or in between, or less than 1 mm or more than 5 mm.

In any of the embodiments, wherein layers of polymeric material blends are consolidated, multiple layers can be used. The layers can be uniform or can vary in thickness along the direction perpendicular to the compression direction. Layers can comprise polymeric materials in different forms, such as powder or pellet. For example, a 1-mm thick UHMWPE/GS pellet can be prepared by blending UHMWPE with GS, pelletizing the blend at approximately 20 MPa and at room temperature for 15 minutes. The pellet can then be subjected to vacuum treatment for 18 hours at 45° C. Adequate amount of UHMWPE powder without additives can then be put in the pre-heated mold, the vacuum-treated pellet comprising UHMWPE with GS can be placed on top of the UHMWPE powder and the two layers be consolidated by compression molding at around 170° C. and approximately 20 MPa of pressure.

The consolidated polymeric materials, for example, consolidated blends of GS/UHMWPE can be molded in the shape of the intended medical device or can be molded in a shape close to the intended medical device shape in one or more dimensions and can be machined into the final intended shape of a medical device made of polymeric materials made with methods described in any of the embodiments.

In some embodiments, discoloration of GS/UHMWPE samples prepared by compression molding, may arise from the Maillard Reactions or caramelization of the additive(s), for instance the caramelization of Gentamicin sulfate. In some embodiments, discoloration during consolidation of the polymeric material blend, for example GS/UHMWPE, can be minimized by dehydration methods described herein and/or by using additives in the polymeric material blend such as calcium chloride. In the latter embodiment the calcium chloride acts as a desiccant or a dehydration agent.

In some embodiments, the polymeric material blend, either in powder form or in pellet form, can be placed in the mold for consolidation. The polymer material blend can be covered by a layer of desiccant or dehydration agent, either in powder form or in pellet form, prior to consolidation. The two layers are pressurized and heated for consolidation. The top layer, that is the desiccant or dehydration agent layer, actively reduces the water content in the polymer material blend and minimize discoloration during consolidation.

In some embodiments, blended GS/UHMWPE can be microwave treated to dehydrate the GS/UHMWPE blend. The duration of microwave treatment can be anywhere between 1 second to several hours or more. In any of the embodiments, the power of the microwave used can be 50 W, 60 W, 70 W, 80 W, 90 W, 100 W, 200 W, 300 W, 400 W, 500 W, 600 W, 700 W, 800 W, 900 W, 1000 W, 1100 W, 1200 W or more than 1200 W or less than 50 W or in between those wattages.

In some embodiments, blended GS/UHMWPE can be vacuum treated to maintain an environment with reduced oxygen and humidity. Any vacuum treatment can be at 0.000001 atm to 0.9999 atm, more preferably 0.001 atm to 0.2 atm, most preferably about 0.001 to 0.1 atm. Vacuum treatment can be at 0° C., 5° C., 10° C., 20° C., 25° C., 30° C., 40° C., 50° C., 60° C., 70° C., 80° C., 90° C., 100° C., 110° C., 120° C., 130° C., 145° C., 150° C., 160° C., 170° C. or above or at any temperature in-between. Preferably at 100° C.-120° C. More preferably at 70° C.-90° C. Most preferably at 40° C.-60° C. Vacuum treatment can be for 1 min to several days.

In some embodiments, GS/UHMWPE blend can be completely consolidated by compression molding. Or GS/UHMWPE blend can be completely consolidated by isostatic pressing. Any of the compression molding can be at 10 MPa, 20 MPa, 40 MPa, less than 10 MPa or more than 40 MPa. Compression molding can be at 0° C., 5° C., 10° C., 20° C., 25° C., 30° C., 40° C., 50° C., 60° C., 70° C., 80° C., 90° C., 100° C., 110° C., 120° C., 130° C., 140° C., 150° C., 160° C., 170° C., 180° C., 190° C., 200° C., 210° C., 220° C., 230° C., 240° C., 250° C., 260° C., 270° C., 280° C., 290° C., 300° C., 310° C., 3 Preferably at 130° C.-140° C. More preferably at 140° C.-150° C. Most preferably at 160° C.-180° C. Any of the compression molding can be for less than 1 min to several days. In any of the embodiments, isostatic pressing can be applied cold or hot. For example, the GS/UHMWPE blend can be consolidated to result in an approximately 3-mm thick sample by compression molding at 170° C., at a pressure setpoint of 20 MPa for 8 minutes in a pre-heated mold or to result in an approximately 17-mm thick sample can be consolidated by compression molding at 170° C., at a pressure setpoint of 20 MPa for 30 minutes in a pre-heated mold.

In some embodiments, GS/UHMWPE blend can be further blended with a sulfite compound. The sulfite compound can be sodium sulfite or sodium bisulfite or sodium metabisulfite or potassium metabisulfite or calcium sulfite or calcium hydrogen sulfite or potassium hydrogen sulfite. The sulfite compound can be dry or wet blended with GS/UHMWPE. The sulfites can be used to minimize the Maillard reaction.

In some embodiments, a GS/UHMWPE blend can be further blended with pyridoxine hydrochloride (vitamin B6). The pyridoxine hydrochloride (vitamin B6) compound can be dry or wet blended with GS/UHMWPE.

### DETAILED DESCRIPTION OF THE INVENTION

The term ‘dry blending’ refers to blending polymeric material powder with additive powder in dry state. Some polymeric materials and some additives are sourced in powder form. The blending of the polymeric material(s) and the additive(s) can be done as is or after controlling the particle size distribution of the polymeric material(s) and/or the additive(s). One way of controlling the particle size distribution is by passing the powder(s) through one or more sieves. While passing the powder(s) through the sieve(s) one can use mechanical agitation and/or mechanical attrition to break down clumps. Blending, dry or wet blending, can be done in a blender or a mixer known in the art such as an octagonal blender, mass mixer, V-type blender, double cone blender, cold screw blender, double planetary mixer, static mixer, paddle mixer, drum mixer, agitator, or such. Blending, dry or wet blending, can be carried out in air, enough gas, partial vacuum or mixtures thereof.

The term ‘wet blending’ refers to blending polymeric material powder with additive(s) in liquid state. In some embodiments, the polymeric material can be in liquid state. The liquid state of the polymeric material and/or the additive can also be achieved by dissolving them in a solvent. It can be desirable to remove the solvent after wet blending. Removal of solvent can be done by dehydration methods described here. In one embodiment, polymeric material powder can be blended with liquid additive(s).

The term ‘discoloration’ refers to change in the color of the polymeric material blend at any state of its processing in any of the embodiments. The discoloration may or may not be uniform. Discoloration may be caused by the caramelization of one of the additives. For instance, caramelization of gentamicin sulfate in GS/UHMWPE blend results in darker colors, mostly caused by water retained in the polymeric material blend.

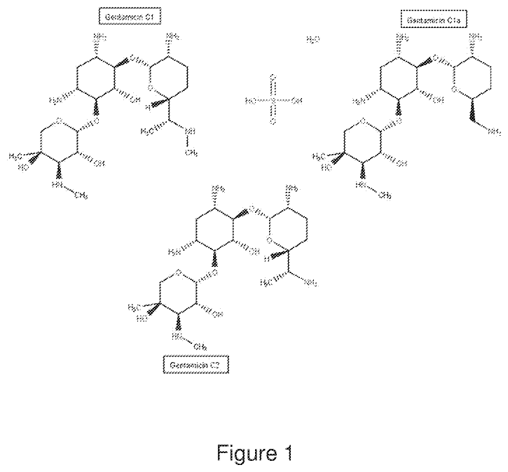

The term ‘gentamicin sulfate’ refers to what is known in the art as an aminoglycoside type antibiotic that is effective against bone infections, endocarditis, pelvic inflammatory disease, sepsis, urinary tract infections, pneumonia, meningitis. Its mechanism of action is comprised of inhibiting bacterial protein synthesis. It is potent against mostly Gram-negative bacteria including Pseudomonas, Proteus, Escherichia coli, Klebsiella pneumoniae, Enterobacter aerogenes, Serratia and Gram-positive Staphylococcus. Gentamicin has three main isomers that are denoted as C₁a, C₁, C₂ and the gentamicin sulfate that was used in the examples provided herein comprised all three isomers (FIG. 1). It is stable as a free base in solvated form and it is also stable as a hydrated salt in powder form (FIG. 1). Its concentrations in polymeric matrices such as bone cement can be represented based on the molecular conversion to the free base form.

The term ‘vancomycin HCl’ refers to what is known in the art as a glycopeptide type antibiotic.

The term ‘vacuum treatment’ or ‘vacuum’ refers to maintaining materials in an environment where the pressure is lower than ambient pressure. The vacuum chamber may contain various combinations of atmospheric gases such as oxygen, nitrogen, carbon dioxide, argon, water vapor. Vacuum treatment can be performed at any pressure from approximately 0 in. Hg (<0.0001 atm) to 30 in. Hg (approximately 1 atm or ambient pressure) and at any temperature between −100° C. and 300° C. or above. The duration of the vacuum treatment can be as short as 1 second and as long as several months or years, more preferably 1 minute to 24 hours, most preferably 2 hours to 12 hours. Applying vacuum refers to lowering the pressure below ambient pressure. In some embodiments, applying vacuum also means that the pressure is reduced in the vacuum chamber and the pressure is brought back up to a certain level by pumping an inert gas, such as nitrogen, argon, carbon dioxide, and the pressure is again lowered blood pressure, which fluctuation in pressure can be repeated as many times as desired.

The term ‘dehydration’ refers to removal of water. Dehydration can be by heating. Dehydration can be by heating in an oven. Dehydration can be by heating in a microwave oven. Dehydration can be carried under atmospheric pressure, under pressures lower than atmospheric pressure, under partial vacuum, with or without active heating. The duration of dehydration can be anywhere between 1 second to several hours or more. Heating can be carried out by reading heating, convection heating, microwave heating. Vacuum dehydration can be under partial vacuum at 0.000001 atm to 0.9999 atm, more preferably 0.001 atm to 0.2 atm, most preferably about 0.001 to 0.1 atm. Vacuum dehydration can be at 0° C., 5° C., 10° C., 20° C., 25° C., 30° C., 40° C., 50° C., 60° C., 70° C., 80° C., 90° C., 100° C., 110° C., 120° C., 130° C., 145° C., 150° C., 160° C., 170° C. or above or at any temperature in-between. Vacuum treatment can be for 1 min to several days. The duration of microwave treatment can be anywhere between 1 second to several hours or more. In any of the embodiments, the power of the microwave used can be 50 W, 60 W, 70 W, 80 W, 90 W, 100 W, 200 W, 300 W, 400 W, 500 W, 600 W, 700 W, 800 W, 900 W, 1000 W, 1100 W, 1200 W or more than 1200 W or less than 50 W or in between those wattages. Dehydration steps described above can also be used for other purposes to precondition polymeric material blend before consolidation. ‘Dehydration’ is also called vacuum-treatment in some embodiments.

Dehydration can also be done by lyophilization. The term ‘lyophilization’ refers to freeze-drying for removal of water. The water can be removed by sublimation. The material can be frozen and pressure can be reduced to sublimate the water. With freezing and lower pressure heat can also be added to accelerate the sublimation. Lyophilization can be done with the polymeric material(s), additive(s), and/or polymeric material blend at any stage of fabrication described in any of the embodiments herein.

The term ‘desiccant’ and ‘dehydration agent’ are used interchangeably and they refer to additive(s) and/or materials that are hygroscopic and are able to remove water from their surroundings. Examples of desiccants are inorganic and organic desiccants and polymers such as Aluminum oxide, Ammonium alginate, Ammonium chloride, Bentonite, Benzalkonium chloride, Boric acid, Butylene glycol Butylparaben, Calcium acetate, Calcium chloride, Calcium sulfate, Carboxymethylcellulose calcium, Carboxymethylcellulose sodium, Chlorhexidine hydrochloride, Citric acid monohydrate, Colloidal silicon dioxide, Docusate sodium, Edetic acid, Lecithin, Magnesium oxide, Potassium citrate, Potassium hydroxide, Sorbitol, Zeolites, Polymeric Additives, Polyacrylic acid (carbomer), Carboxymethylcellulose calcium, Carboxymethylcellulose sodium, Carrageenan, Cellulose (microcrystalline), Cellulose acetate, Chitosan, Copovidone (PVP-co-PVAc), Crospovidone, Ethylcellulose, Hydroxyethyl cellulose, Hydroxyethylmethyl cellulose, Hydroxypropyl cellulose, Hypromellose (Cellulose hydroxypropyl methyl ether), Hypromellose acetate succinate, Imidurea, Maleic Anhydride Copolymers, Poly(acrylic acid), Polymethacrylate and Other Acrylic Polymers, Polyethylene-b-polyethylene glycol, Polyethylene-co-polyacrylic acid, Poloxamer, Polycarbophil, Poly(methyl vinyl ether/maleic anhydride), Polysorbate, Poly(2-oxazoline) and Polyethylenimine (PEI), Poly(vinylpyrrolidone) (PVP) and Copolymers, Poly(vinyl alcohol) (PVA) and Copolymers, Poly (vinyl alcohol)-co-polyethylene, Sodium alginate, and Starch.

The polymeric material can also be blended with other additives that are not described as therapeutic agents. For example, GS can be blended with UHMWPE which can be pre-blended with an antioxidant such as vitamin E or a mixture of antioxidants. The percentage by weight of therapeutic agent(s) in the therapeutic agent blended polymeric material can be from 0.001 wt % to 50 wt % or 1%, 2%, 3%, 4%, 5%, 6%, 7%, 8%, 9%, 10% or more or less than 1% or in between those percentages. In the case of a polymeric material pre-blended with an antioxidant, the antioxidant concentration can be from 0.001 wt % to 50 wt % or 1%, 2%, 3%, 4%, 5%, 6%, 7%, 8%, 9%, 10% or more or less than 1% or in between those percentages.

The term “Maillard Reactions” refers to the cascade of reactions initiated by the reaction between carbonyl and amine groups (first described in L.-C. Maillard, Action des acides amines sur les sucres: formation des melanoidines par voie methodique, C.R. Hebd. Seances Acad. Sci., 1912, 154, 66-68). The carbonyl group may be present in a sugar molecule and the amine group may be present in an amino acid, a peptide, a protein or a carbohydrate or its derivative. Maillard Reactions can be initiated in the presence of an acid and can be more favorable when the temperature of the milieu increases. Maillard Reactions may yield Amadori Compounds such as enol and keto forms of N-substituted 1-amino-2-deoxy-2-ketose molecules or may yield Schiff Bases or furfural or its derivatives such as hydroxymethylfurfural or aldehyde derivatives or aldol derivatives or melanoidin derivatives (K. Eichner, M. Reutter, and R. Wittmann, Detection of Amadori compounds in heated foods, in Thermally Generated Flavors: Maillard, Microwave, and Extrusion Process, T. H. Parliment, M. J. Morello, R. J. McGorrin (eds), American Chemical Society, Washington, D C, 1994, 42-54). In some instances, the Maillard Reaction term can be interchangeably used with the term browning or discoloration. The use of therapeutics for the compression molded implant material, such as aminoglycoside antibiotic and glycopeptide antibiotics may give rise to Maillard Reactions. Discoloration of blends of UHMWPE with aminoglycosides or glycopeptides during processing may be partly due to Maillard reactions (P. M. T. de Kok and E. A. E. Rosing, Reactivity of peptides in Maillard reaction, in Thermally Generated Flavors: Maillard, Microwave, and Extrusion Processes, T. H. Parliment, M. J. Morello, and R. J. McGorrin (eds), American Chemical Society, Washington, D C, 1994, 158-179). The methods of making consolidated polymeric materials can decrease Maillard reactions of aminoglycosides or glycopeptides in the processing of blends of UHMWPE. The term “caramelization” refers to the reaction of sugars upon heating (E.-H. Ajandouz, L. S. Tchiakpe, F. Dalle Ore, A. Benajiba, and A. Puigserver, Effects of pH on caramelization and Maillard reaction kinetics in fructose-lysine model systems, J. Food Sci., 2001, 66, 926-931). As they are sugar derivatives, aminoglycoside and glycopeptide antibiotics may undergo caramelization at elevated temperatures. The methods of making consolidated polymeric materials can decrease caramelization of aminoglycosides and glycopeptides in the processing of blends of polymeric material. For instance, dehydration the aminoglycoside or the glycopeptide antibiotics can help decrease the caramelization and hence decrease discoloration.

“Polymeric materials” or “polymers” refers to what is known in the art as chemical entities comprising chains of repeating subunits. They can include structural subunits different from each other. Such polymers can be di- or tri- or multiple unit-copolymers, alternating copolymers, star copolymers, brush polymers, grafted copolymers or interpenetrating polymers. They can have a low solvent presence during processing and use such as thermoplastics or can include a large amount of solvent such as hydrogels. Polymeric materials also include synthetic polymers, natural polymers, blends and mixtures thereof. Polymeric materials also include degradable and non-degradable polymers.

The products and processes provided herein also apply to various types of polymeric materials, for example, any polypropylene, any polyamide, any polyether ketone, or any polyolefin, including high-density-polyethylene, low-density-polyethylene, linear-low-density-polyethylene, ultra-high molecular weight polyethylene (UHMWPE), copolymers or mixtures thereof. The products and processes provided herein also apply to various types of hydrogels, for example, poly(vinyl alcohol), polyethylene glycol), polyethylene oxide), poly(acrylic acid), poly(methacrylic acid), poly(acrylamide), copolymers or mixtures thereof, or copolymers or mixtures of these with any polyolefin. Polymeric materials, as used herein, also applies to polyethylene of various forms, for example, resin, powder, flakes, particles, powder, or a mixture thereof, or a consolidated form derived from any of the above. Polymeric materials, as used herein, also applies to hydrogels of various forms, for example, film, extrudate, flakes, particles, powder, or a mixture thereof, or a consolidated form derived from any of the above. Polymeric materials, as used herein, also applies to the types of polymeric materials described above that are also blended with additive(s). For example, a polymeric material could be, UHMWPE blended with gentamicin sulfate, or UHMWPE blended with gentamicin sulfate and an antioxidant such as vitamin E, or UHMWPE blended with vancomycin HCl with or without an antioxidant, or UHMWPE blended with gentamicin sulfate and vancomycin HCl with or without an antioxidant. By polymeric material is meant a polymeric material with or without any additives.

By “medical device”, what is meant is an instrument, apparatus, implement, machine, implant or other similar and related article intended for use in the diagnosis, treatment, mitigation, cure, or prevention of disease in humans or other animals. An “implantable device” is a medical device intended to be implanted in contact with the human or other animal for a period of time. “Implant” refers to an “implantable medical device” where a medical device, can be placed into contact with human or animal skin or internal tissues for a prolonged period of time, for example at least 2 days or more, or at least 3 months or more, or at least six months or more, or permanently. Implants can be made out of metals, ceramic, polymers or combinations thereof. They can also comprise fluids or living tissues in part or in whole. An “implant” can refer to several components together serving a combined function such as “total joint implant” or it can refer to a single solid form such as an “acetabular cup” as a part. The term ‘medical implant’ refers to a medical device made for implantation in a living body, for example and animal or human body. The medical implants include but are not limited to acetabular liners, tibial inserts, glenoid components, patellar components, and other load-bearing, articular components used in total joint surgery. While medical implants can be load-bearing to some extent some bear more load than others. For instance a tibial insert bears more load than a man-hole cover implant used to cover screw holes in acetabular shells. The term “permanent device” refers to what is known in the art that is intended for implantation in the body for a period longer than several months. Permanent devices include medical implants or devices, for example, acetabular liner, shoulder glenoid, patellar component, finger joint component, ankle joint component, elbow joint component, wrist joint component, toe joint component, bipolar hip replacement component, tibial knee insert, tibial knee inserts with reinforcing metallic and polyethylene posts, intervertebral discs, sutures, tendons, heart valves, stents, and vascular grafts. The term “medical implant” refers to what is known in the art as a device intended for implantation in animals or humans for short- or long-term use. The medical implants provided herein comprise medical devices including acetabular liner, shoulder glenoid, patellar component, finger joint component, ankle joint component, elbow joint component, wrist joint component, toe joint component, bipolar hip replacements, tibial knee insert, tibial knee inserts with reinforcing metallic and polyethylene posts, intervertebral discs, sutures, tendons, heart valves, stents, and vascular grafts, fracture plates. Medical implants can also include ‘temporary devices’, which are intended for implantation but not permanently. For example, articulating spacers placed in the joint after revision joint arthroplasty are intended to stay in the body for up to 6 months.

The term “blending” refers to what is known in the art; that is, mixing of different components, often liquid and solid or solid and solid to obtain a mixture of components. Blending generally refers to mixing of a polymeric material in its pre-consolidated form with an additive. If both constituents are solid, blending can be done by using other component(s) such as a liquid to mediate the mixing of the two components, after which the liquid can be removed for instance by evaporation. If the additive is liquid, for example a-tocopherol, then the polymeric material can be mixed with large quantities of the liquid. This high concentration blend can be diluted down to desired concentrations with the addition of lower concentration blends or virgin polymeric material without the additive to obtain the desired concentration blend. This technique also results in improved uniformity of the distribution of the additive in the polymeric material. Methods of blending additives into polymeric material are described, for example in U.S. Pat. Nos. 7,431,874, 9,168,683, 8,425,815, 9,273,189, and WO2007/024684A2 (Muratoglu et al.). Blending of different additives can be done sequentially or simultaneously, using the same or different methods of incorporation. For example, blending vitamin E with UHMWPE resin powder can be performed with the aid of a solvent such as isopropanol and drying. This blend can further be blended with solid gentamicin sulfate powder, resulting in a vitamin E and GS-blended UHMWPE. A polymeric material incorporated with an additive by blending is termed an “additive-blended” polymeric material. For example, if the blending of polymeric material is done with a therapeutic agent(s), the resulting blend can be described as a therapeutic agent-blended polymeric material or a blend of the therapeutic agent/polymeric material. For example, if the therapeutic agent is gentamicin sulfate (GS) and the polymeric material is UHMWPE, then a blend can be described as GS-blended UHMWPE or a blend of GS/UHMWPE or a GS/UHMWPE blend.

The term “Diffusion” refers to what is known in the art; that is, the net movement of molecules from an area of high concentration to an area of low concentration. In these embodiments, it is defined to be interchangeably used with ‘doping by diffusion’. The term “doping” refers to a general process well known in the art (see, for example, U.S. Pat. Nos. 6,448,315 and 5,827,904), that is introducing additive(s) to a material. Doping may also be done by diffusing an additive into the polymeric material by immersing the polymeric material by contacting the polymeric material with the additive in the solid state, or with a bath of the additive in the liquid state, or with a mixture of the additive in one or more solvents in solution, emulsion, suspension, slurry, aerosol form, or in a gas or in a supercritical fluid. The doping process by diffusion can involve contacting a polymeric material, medical implant or device with an additive, such as vancomycin or gentamicin, for about an hour up to several days, preferably for about one hour to 24 hours, more preferably for one hour to 16 hours. The doping time can be from a second to several weeks, or it can be 1 minute to 24 hours, or it can be 15 minutes to 24 hours. The environment for the diffusion of the additive (bath, solution, emulsion, paste, slurry and the like) can be heated to room temperature or up to about 200° C. and the doping can be carried out at room temperature or up to about 200° C. For example, when doping a polymeric material by an antioxidant, the medium carrying the antioxidant can be heated to 100° C. and the doping can be carried out at 100° C. Similarly, when doping a polymeric material with therapeutic agent(s), the medium carrying the therapeutic agent(s) can be cooled or heated. Or the doping can be carried out at 5, 10, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 230, 240, 250, 260, 270, 280, 290, 300, 320 and 340° C., and any value therebetween. A polymeric material incorporated with an additive by diffusion in such a way is termed an “additive-diffused” polymeric material. If the additive is a therapeutic agent, a polymeric material incorporated with the additive is termed a “therapeutic agent-diffused” polymeric material. Diffusion of additives such as antioxidants by high temperature doping and homogenization methods are described in Muratoglu et al. (U.S. Pat. Nos. 7,431,874 and 9,370,878), the teachings of which are hereby incorporated by reference. Doping can also be done using polymeric material in unconsolidated form, for example, powder, flake or pellets and can thus be used at times interchangeably with blending.

The term ‘compression molding’ refers to method of consolidation by molding. In the case of UHMWPE, consolidation is most often performed by “compression molding”. In some instances, consolidation can be interchangeably used with compression molding. The molding process generally involves: (i) heating the polymeric material to be molded, (ii) pressurizing the polymeric material while heated, (iii) keeping at elevated temperature and pressure, and (iv) cooling down under pressure and (v) releasing pressure. Typically the consolidation can be carried out by pressurizing the heated polymeric material inside a mold for the consolidated polymeric material to obtain the shape of the mold.

The term “additive” refers to an antioxidant or a therapeutic agent.

The term “therapeutic agent” refers to what is known in the art, that is, a chemical substance or a mixture thereof capable of eliciting a healing reaction from the human body. A therapeutic agent can be referred to also as a “drug” in this application. The therapeutic agent can elicit a response that is beneficial for the human or animal. Examples of therapeutic agents are antibiotics, anti-inflammatory agents, anesthetic agents, anticoagulants, hormone analogs, contraceptives, vasodilators, vasoconstrictors, or other molecules classified as drugs in the art. A therapeutic agent can sometimes have multiple functions. One or more therapeutic agents can be utilized according to the invention.

Examples of therapeutic agents are antimicrobials such as but not limited to Gatifloxacin, gemifloxacin, moxifloxacin, levofloxacin, pefloxacin, ofloxacin, ciprofloxacin, aztreonam, meropenem, imipenem, ertapenem, doripenem, piperacillin, Piperacillin-Tazobactam, Ticarcilin-Clavulanic acid, Ticarcillin, ampicillin-sulbactam, amoxicillin-clavulanic acid, ampicillin-amoxicillin, cioxacillin, nafcillin, oxacillin, methicillin, penicillin V, penicillin G, cefpodox, cefdinir, cefditoren, ceftibuten, cefixime, cefuroxime axetil, cefprozil, cefaclor, loracarbef, cephalexin, cefadroxil, cefepime, ceftazidime, ceftaroline, ceftriaxone, ceftizoxime, cefotaxime, cefuroxime, cefuroxime acetil, cefaclor-CD, cefoxitin, cefotetan, cefazolin, cefdinir, cefditoren pivoxil, cefixime, cefpodoxime proxetil, ceftobiprole, colistimethate, linezolid, quinupristin-dalfopristin, metronidazole, rifampin, fosfomycin, nitrofurantoin, TMP-SMX, trimethoprim, fusidic acid, telavancin, teicoplanin, Vancomycin HCl, vancomycin free base, daptomycin, tigecycline, minocycline, doxycycline, telithromycin, clarithromycin, azithromycin, azithromycin ER, erythromycin, clindamycin, chloramphenicol, amikacin, tobramycin, gentamycin, aztreonam, kanamycin, tetracycline, tetracycline HCl, polymyxin B, rifaximin, tigecycline, amphotericin B, fluconazole, itraconazole, ketoconazole, posaconazole, voriconazole, anidulafungin, caspofungin, flucytosine, micafungin, capreomycin, cycloserine, ethambutol, ethionamide, isoniazid, para-aminosalocylic acid, pyrazinamide, rifabutin, rifapentine, streptomycin, albendazole, artemether/lumefantrine, atovaquone, dpasone, ivermectin, mefloquine, miltefosine, nitazoxanide, proguanil, pytimethamine, praziquantel, tinidazole. Antiviral such as acyclovir, cidofovir, probenecid, entecavir, famciclovir, foscarnet, ganciclovir, oseltamivir, peramivir, ribavirin, rimantadine, telbiudine, valacyclovir, valgancciclovir, abacavir, atazanavir, darunavir, delaviridine, didanosine, efavirenz, emtricitabine, enfuvirtide, etravirine, fosamprenavir, indinavir, lamivudine, lopinavir, maraviroc, nelfinavir, nevirapine, raltegravir, ritonavir, sasquinavir, stavudine, tenofovir, tipranavir, zidovudine. Antifibrinolytics such as 8-aminocaproic acid, tranexamic acid, lysine, aprotinin. Antineoplastics such as mechlrethamine, phenylalanine mustard, chlorambucil, cyclophosphamide, busulfan, triethylene-thiophosphoramide, carmustine, DTIC, methotrexate, 5-fluorouracil, 6-mercaptopurine, vincristine, procarbazone, prednisone, acivicin, aclarubicin, acodazole, acronine, adozelesin, alanosine, alpha-Tgdr, altretamine, ambomycin, amentantrone acetate, aminopterin, aminothiadiazole, amsacrine, anguinide, aniline mustard, anthramycin, azaribine, 5-aza-2′Deoxycytidine, 8-azaguanine.

Other examples of therapeutic agents are analgesics. The term ‘analgesic’ refers to a therapeutic agent used in the relief of pain. The mechanism by which pain is relieved can be different with different analgesics. Examples are compounds such as but not limited to bupivacaine, ropivacaine, lidocaine or non-steroid anti-inflammatories such as ketorolac, meloxicam, salicylic acid, ibuprofen, diclofenac sodium, tolfenamic acid. Blends of polymeric material with these analgesic therapeutic compounds can be made using any of the methods described herein.

The term “antioxidant” refers to what is known in the art as (see, for example, WO 01/80778, U.S. Pat. No. 6,448,315). Alpha- and delta-tocopherol; propyl, octyl, or dedocyl gallates; lactic, citric, ascorbic, tartaric acids, and organic acids, and their salts; orthophosphates, lycopene, tocopherol acetate are generally known form of antioxidants. Antioxidants are also referred as free radical scavengers, include: glutathione, lipoic acid, vitamins such as ascorbic acid (vitamin C), vitamin B, vitamin D, vitamin-E, tocopherols (synthetic or natural, alpha-, gamma-, delta-), acetate vitamin esters, water soluble tocopherol derivatives, tocotrienols, water soluble tocotrienol derivatives; melatonin, carotenoids, including various carotenes, lutein, pycnogenol, glycosides, trehalose, polyphenols and flavonoids, quercetin, lycopene, lutein, selenium, nitric oxide, curcuminoids, 2-hydroxytetronic acid; cannabinoids, synthetic antioxidants such as tertiary butyl hydroquinone, 6-amino-3-pyrodinoles, butylated hydroxyanisole, butylated hydroxytoluene, ethoxyquin, tannins, propyl gallate, other gallates, Aquanox family; Irganox® and Irganox® B families including Irganox® 1010, Irganox® 1076, Irganox® 1330; Irgafos® family including Irgafos® 168; phenolic compounds with different chain lengths, and different number of OH groups; enzymes with antioxidant properties such as superoxide dismutase, herbal or plant extracts with antioxidant properties such as St. John's Wort, green tea extract, grape seed extract, rosemary, oregano extract, mixtures, derivatives, analogues or conjugated forms of these. Antioxidants/free radical scavengers can be primary antioxidants with reactive OH or NH groups such as hindered phenols or secondary aromatic amines, they can be secondary antioxidants such as organophosphorus compounds or thiosynergists, they can be multifunctional antioxidants, hydroxylamines, or carbon centered radical scavengers such as lactones or acrylated bis-phenols. The antioxidants can be selected individually or used in any combination.

Irganox®, as described herein refers to a family of antioxidants manufactured by Ciba Specialty Chemicals. Different antioxidants are given numbers following the Irganox® name, such as Irganox® 1010, Irganox® 1035, Irganox® 1076, Irganox® 1098, etc. Irgafos® refers to a family of processing stabilizers manufactured by Ciba Specialty Chemicals. Irganox® family has been expanded to include blends of different antioxidants with each other and with stabilizers from different families such as the Irgafos family. These have been given different initials after the Irganox® name, for instance, the Irganox® HP family are synergistic combinations of phenolic antioxidants, secondary phosphate stabilizers and the lactone Irganox® HP-136. Similarly, there are Irganox® B (blends), Irganox® L (aminic), Irganox® E (with vitamin E), Irganox® ML, Irganox® MD families. Herein we discuss these antioxidants and stabilizers by their tradenames, but other chemicals with equivalent chemical structure and activity can be used. Addition, these chemicals can be used individually or in mixtures of ant composition. Some of the chemical structures and chemical names of the antioxidants in the Irganox® family are listed in Table 1.

TABLE 1
Chemical names and structures of some antioxidants trademarked under the Irganox ® name.

| Trade | Chemical | |
|---|---|---|
| name | name | Chemical Structure |
| Irgano x ® 1010 | Tetrakis [methylene (3,5-di-tert- butyl- hydroxy- hydro- cinnamate)] methane | 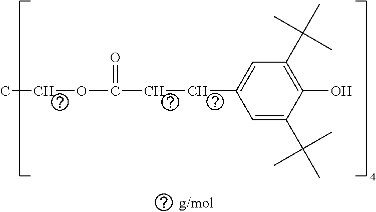 |
| Irgano x ® 1035 | Thiodiethylene bis[3-[3,5-di- tert-butyl-4- hydroxy- phenyl] propionate] | 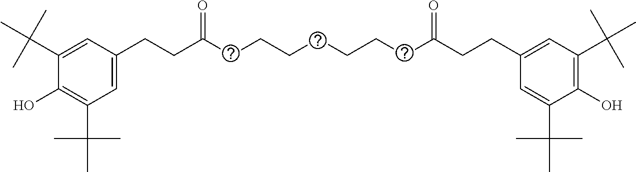 [SMILES: CC(C)(C)c1cc(CCC(=O)CCCCCCCC(=O)CCc2cc(C(C)(C)C)c(O)c(C(C)(C)C)c2)cc(C(C)(C)C)c1O] |
| Irgano x ® 1076 | Octadecyl 3,5-di- tert-butyl-4- hydroxyl- hydro- cinnamate | 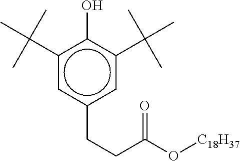 |
| Irgano x ® 1098 | N,N′-hexane- 1,6-diylbis (3-(3,5- di-tert- butyl-4- hydroxy- phenyl- propion- amide)) | |
| Irgano x ® 1135 | Benzene- propanoic acid,3,5- bis(1,1- dimethyl- ethyl)-4- hydroxy- .C7-C9 branched alkyl esters | 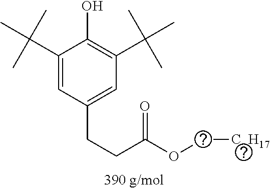 |
| Irgano x ® 1330 | 1,3,5-tris (3,5-di- tert-butyl-4- hydroxy- benzyl)- 2,4,6- trimethyl- benzene | 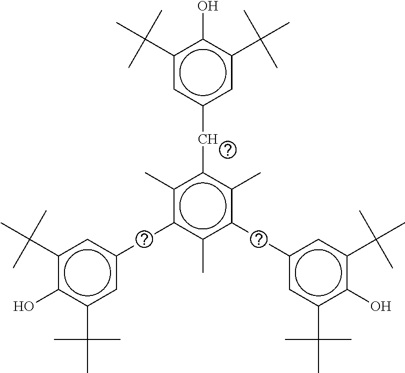 |
| Irgano x ® 1520 | | 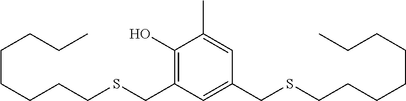 [SMILES: CCCCCCCCSCc1cc(C)c(O)c(CSCCCCCCCC)c1] |
| Irgano x ® 1726 | 2,4-bis (dodecyl- thiomethyl)- 6-methyl- phenol | 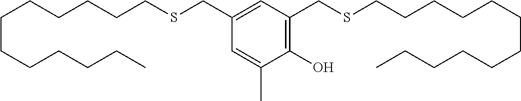 [SMILES: CCCCCCCCCCCCSCc1cc(C)c(O)c(CSCCCCCCCCCCCC)c1] |
| Irgano x ® 245 | Triethylene glycol bis (3-tert- butyl-4- hydroxy- 5-methyl- phenyl) propionate | 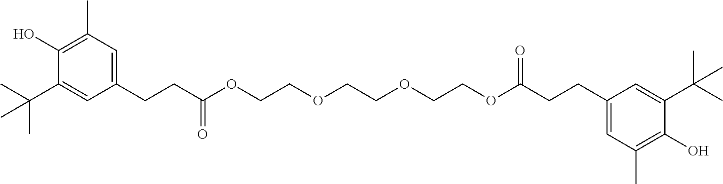 [SMILES: Cc1cc(CCC(=O)OCCOCCOCCOC(=O)CCc2cc(C)c(O)c(C(C)(C)C)c2)cc(C(C)(C)C)c1O] |
| Irgano x ® 3052 | 2,2′- methylenebis (4-methyl- 6-tert- butylphenol) mono- acrylate | 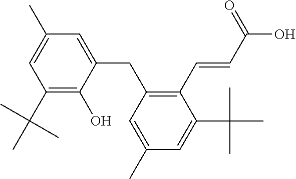 [SMILES: Cc1cc(Cc2cc(C)cc(C(C)(C)C)c2/C=C/C(=O)O)c(O)c(C(C)(C)C)c1] |
| Irgano x ® 3114 | 1,3,5-TRis (3,5-di-tert- butyl-4- hydroxy- benzyl)- 1,3,5- triazine- 2,4,6 (1H,3H,5H)- trione | 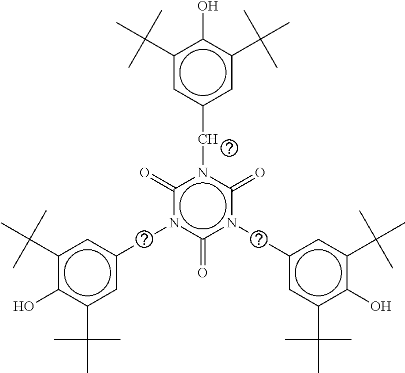 |
| Irgano x ® 5057 | Benzenamine, N-phenyl-, reaction products with 2,4,4- trimethyl- pentene | 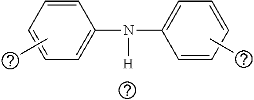 |
| Irgano x ® 565 | 2,4-bis (octylthio)- 6-(4- hydroxy- 3,5-di-tert- butylanilino)- 1,3,5- triazine | 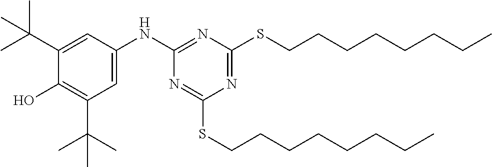 [SMILES: CCCCCCCCSc1nc(Nc2cc(C(C)(C)C)c(O)c(C(C)(C)C)c2)nc(SCCCCCCCC)n1] |
| Irgano x ® HP-136 | 5,7-di-t- butyl- 3-(3,4 di- methyl- phenyl)- 3H- benzofuran- 2-one | 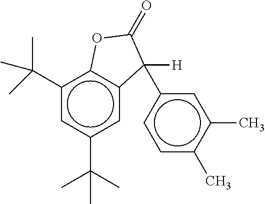 |
| Irgafo s ® 168 | Tris(2,4-di-tert- butylphenyl) phospite | 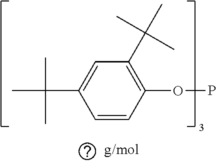 |

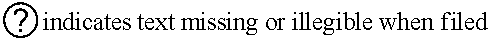

The term “consolidation” refers generally to processes used to convert the polymeric material resin, particles, flakes, i.e. small pieces of polymeric material, or polymeric material blend into a mechanically integral large-scale solid form, which can be further processed, by for example machining in obtaining articles of use such as medical implants. Methods such as injection molding, extrusion, ram extrusion, compression molding, iso-static pressing (hot or cold), or other methods known in the art can be used. Consolidation of layers of polymeric material having different additives is also described herein.

Consolidation can be performed by “compression molding”. In some instances consolidation can be interchangeably used with compression molding. The molding process generally involves:
i. heating the polymeric material to be molded,
ii. pressurizing the polymeric material while heated,
iii. keeping at temperature and pressure, and
iv. cooling down under pressure and
v. releasing pressure.

Heating of the polymeric material can be done at any rate. Temperature can be increased linearly with time or in a step-wise fashion or at any other rate. Alternatively, the polymeric material can be placed in a pre-heated environment. In some embodiments, the polymeric material can be placed into a mold for consolidation and the process (steps i-v) can be started without pre-heating. The mold for the consolidation can be heated together or separately from the polymeric material to be molded. Steps (i) and (ii), i.e. heating and pressurizing before consolidation can be done in multiple steps and in any order. For example, polymeric material can be pressurized at room temperature to a set pressure level 1, after which it can be heated and pressurized to another pressure level 2, which still may be different from the pressure or pressure(s) in step (iii). Step (iii), where a high temperature and pressure are maintained is the ‘dwell period’ where a major part of the consolidation takes place. One temperature and pressure or several temperatures and pressures can be used during this time without releasing pressure at any point. For example, dwell temperatures in the range of 135 to 350° C. and dwell pressures in the range of 0.1 MPa to 100 MPa or up to 1000 MPa, or any value therebetween, can be used. The dwell time can be from 1 minute to 24 hours, more preferably from 2 minutes to 1 hour, most preferably about 10 minutes. The temperature(s) at step (iii) are termed ‘dwell’ or ‘molding’ temperature(s). The pressure(s) used in step (iii) are termed ‘dwell’ or ‘molding’ pressure(s). The order of cooling and pressure release (step iv) can be used interchangeably. In some embodiments the cooling and pressure release may follow varying rates independent of each other. In some embodiments, consolidation of polymeric resin or blends of the resin with additive(s) are achieved by compression molding. The dwell temperature and dwell time for consolidation can be changed to control the amount of integration.

Also described herein are ‘partial consolidation’ or ‘partially consolidated’ or ‘pre-molded’ or ‘pelletized’ polymeric material, which refers to a state of the polymeric material which is less integrated than a ‘completely consolidated’ or ‘consolidated’ form of the polymeric material. The extent of integration can be quantified, for example, by measuring the elongation at break of the polymeric materials after consolidation. In general, a lower elongation at break indicates a less integrated or less consolidated state for the same type of polymeric resin. In general, a partially consolidated polymeric material may not have the desired properties to be used as a final product and should be further integrated or processed or consolidated to increase its state of consolidation and/or to reduce its porosity. In pre-molding or pelletization, temperature and pressure steps can be applied separately. Also in pre-molding or pelletization, ambient temperature can be used. Also in pre-molding or pelletization, ambient pressure, partial pressure below ambient pressure to reduce oxygen concentration, or vacuum can be used. Also, the pre-molding or pelletization step can be performed in air or in inert gas or in vacuum or partial vacuum or a mixture thereof. The inert gas can be argon, helium, nitrogen or a mixture thereof. For example, dwell temperatures in the range of 0° C. to 350° C. and dwell pressures in the range of 0.001 MPa to 100 MPa or up to 1000 MPa can be used. The dwell time can be from 1 minute to 24 hours or longer, more preferably from 2 minutes to 1 hour, most preferably about 15 minutes or shorter.

Compression molding can also follow “layering” of different polymeric material; in these instances, it is termed “layered molding”. This refers to consolidating a polymeric material by compression molding one or more of its pre-molded, pelletized and resin forms, which may be in the form of flakes, powder, pellets or the like or consolidated or pre-molded forms in layers. This may be done such that there can be distinct regions in the consolidated form containing different concentrations of additives such as antioxidant(s), and/or therapeutic agent(s). Layering can be done in any method that deposits desired polymeric material in desired locations. These methods may include pouring, scooping, painting, brushing, and/or spraying. This deposition can be aided by materials, templates and such supporting equipment that do not become an eventual part of the consolidated polymeric material. Whenever a layered-molded polymeric material is described and is used in any of the embodiments, it can be fabricated by:

(a) layered molding of polymeric resin powder or blends of polymeric material containing certain additive(s) where one or more layers contain additive(s) and one or more layers do not contain some or all of the additive(s);

(b) molding together of layers of polymeric material containing different or same additive(s) such as therapeutic agent(s), and/or antioxidant(s).

Layering and spatial control of additive concentrations and polymeric material morphology are described in WO2008/092047A1, U.S. Pat. Nos. 9,433,705, and 8,569,395 (Muratoglu et al.), the teachings of which are hereby incorporated by reference.

One or more of the layers can be treated before or during molding by heating, or high temperature melting. Methods of high temperature melting are described in WO2010/096771A2, U.S. Pat. No. 8,933,145 (Oral et al.), the teachings of which are hereby incorporated by reference. In some embodiments, one or more of the layers can be pre-treated by heating, cooling or annealing at a temperature below, close to or above the peak melting temperature of the polymeric material. In some embodiments, one or more of the layers can be pre-molded, pelletized or partially consolidated. In some embodiments, one or more of the layers can be pre-blended with additives such as therapeutic agents or antioxidants. In some embodiments, one or more of the layers can be pre-treated environmentally such as placing in vacuum for a period of time, in inert environments for a period of time, soaking in a solution followed by drying, freeze drying, In some embodiments, one or more of the layers can be pre-treated with microwave radiation.

The layer or layers to be molded can be heated in liquid(s), in water, in air, in inert gas, in supercritical fluid(s) or in any environment containing a mixture of gases, liquids or supercritical fluids before pressurization. The layer or layers can be pressurized individually at room temperature or at an elevated temperature below the melting point or above the melting point before being molded together. The temperature at which the layer or layers are pre-heated can be the same or different from the molding or dwell temperature(s). The temperature can be gradually increased from pre-heat to mold temperature with or without pressure. The pressure to which the layers are exposed to before molding can be gradually increased or increased and maintained at the same level.

During consolidation, different regions of the mold can be heated to different temperatures. The temperature and pressure can be maintained during molding for 1 second up to 1000 hours or longer. During cooling at a certain cooling rate under pressure, the pressure can be maintained at the molding pressure or increased or decreased. After cooling down to about room temperature, the mold can be kept under pressure for 1 second to 1000 hours, or any value therebetween. Or the pressure can be released partially or completely at an elevated temperature.

In some embodiments, the pre-molded polymeric material can be subjected to high temperature melting and blended with additive(s) and subsequently direct compression molded. The direct compression molded polymeric material may be in its final implant shape. In some embodiments certain features of the final implant shape may be machined after direct compression molding.

Compression molding can also be done such that the polymeric material can be directly compression molded onto a second surface, for example a metal or a porous metal to result in an implant or implant preform. This type of molding results in a “hybrid interlocked polymeric material” or “hybrid interlocked medical implant preform” or “hybrid interlocked medical implant”. Molding can be conducted with a second piece, for example a metal that becomes an integral part of the consolidated polymeric article. For example, a combination of antioxidant-containing polyethylene resin, powder, or flake and virgin polyethylene resin, powder or flake can be direct compression molded into a metallic acetabular cup or a tibial base plate. The porous tibial metal base plate can be placed in the mold, antioxidant blended polymeric resin, powder, or flake can be added on top. Prior to consolidation, the pores of the metal piece can be filled with a waxy or plaster substance through half the thickness to achieve polyethylene interlocking through the other unfilled half of the metallic piece. The pore filler can be maintained through the irradiation and subsequent processing (for example peroxide diffusion) to prevent infusion of components in to the pores of the metal. In some embodiments, the article can be machined after processing to shape an implant. In some embodiments, there can be more than one metal piece integral to the polymeric article. The metal(s) may be porous only in part or non-porous. In another embodiment, one or some or all of the metal pieces integral to the polymeric article can be a porous metal piece that allows bone in-growth when implanted into the human body. In an embodiment, the porous metal of the implant can be sealed using a sealant to prevent or reduce the penetration of polymeric material blend into the pores during consolidation of the implant. Preferably, the sealant is water soluble. But other sealants are also used. The final cleaning step that the implant can be subjected to also removes the sealant. Alternatively, an additional sealant removal step can be used. Such sealants as water, saline, aqueous solutions of water soluble polymers such as poly-vinyl alcohol, water soluble waxes, plaster of Paris, or others are used. In addition, a photoresist like SU-8, or other, may be cured within the pores of the porous metal component. Following processing, the sealant may be removed via an acid etch or a plasma etch. In these embodiments, the polymeric material, which can be molded directly onto a second surface to form the hybrid interlocked polymeric material, maybe a pre-molded polymeric material with or without additives. In such embodiments the pre-molded polymeric material may be subjected to high temperature melting and/or radiation crosslinking.

The term “heating” refers to bringing a material to a temperature, generally a temperature above that of its current state. It can also refer to maintaining said temperature for a period of time, that is, in some instances it can be used interchangeably with ‘annealing’. Heating can be done at any rate. The heating rate can be, for example, from 0.001° C./min to 1000° C./min, or any value therebetween, or it can be between 0.1° C./min to 100° C./min, or it can be from 0.5° C./min to 10° C./min, or it can be any rate from 1° C./min to 50° C./min in 1° C. intervals. The heating can be done for any duration. Heating time can be from 0.1 minutes to 100 years or from 1 minute to 24 hours or from 1 minute to 12 hours, or 30 minutes to 10 hours, or 5 hours, or 6 hours, or 8 hours, or any value therebetween. The heating can be done in consecutive steps of heating to different temperatures, where one heating temperature can be above or below the subsequent heating temperature.

The term “cooling” refers to bringing a material to a temperature, generally a temperature below that of its current state. It can also refer to maintaining said temperature for a period of time, that is, in some instances it can be used interchangeably with ‘annealing’. Cooling can be done at any rate. The cooling rate can be from 0.001° C./min to 1000° C./min, or it can be between 0.1° C./min to 100° C./min, or it can be from 0.5° C./min to 10° C./min, or it can be any rate from 1° C./min to 50° C./min in 1° C. intervals, or 2.5° C./min, or any value therebetween. The cooling can be done for any duration. Cooling time can be from 0.1 minutes to 100 years or from 1 minute to 24 hours or from 1 minute to 12 hours, or 30 minutes to 10 hours, or 1 hour, or 2 hours, or 5 hours, or 6 hours, or 8 hours, or any value therebetween.

The term “sterile” refers to a condition of an object, for example, an interface or a hybrid material or a medical implant containing interface(s), wherein the interface is sufficiently sterile to be medically acceptable, i.e., will not cause an infection or require revision surgery. The object, for example a medical implant, can be sterilized using ionizing radiation or gas sterilization techniques. Gamma sterilization is well known in the art. Electron beam sterilization is also used. Ethylene oxide gas sterilization and gas plasma sterilization are also used. Autoclaving is another method of sterilizing medical implants. Exposure to solvents or supercritical fluids for sufficient to kill infection-causing microorganisms and/or their spores can be a method of sterilizing.

The term “surface” refers to any part of the outside of a solid-form material, which can be exposed to the surrounding liquid, gaseous, vacuum or supercritical medium. The surface can have a depth into the bulk of the material (normal to the surface planes), from several microns (pm) to several millimeters. For example, when a ‘surface layer’ is defined, the layer can have a thickness of several nanometers to several microns (pm) to several millimeters. For example, the surface layer can be 100 microns (100 pm) or 500 microns (500 pm) or 1000 microns (1 mm) or 2 mm or it can be between 2 and 5 mm, or any value therebetween. The surface or surfaces can also be defined along the surface planes. For example, a 5 mm wide and 15 mm long oval section of the articulating surface of a tibial knee insert can be defined as a ‘surface’ to be layered with a UHMWPE containing additives. These surfaces can be defined in any shape or size and the definition can be changed at different processing step.

### EXAMPLES

### Example 1. Dry Blending of Gentamicin Sulfate (GS)/UHMWPE

GS (Sigma Aldrich, St. Louis, Mo.) powder was sieved through a 75 pm mesh sieve and the powder that went through the sieve was ground gently with a ceramic pestle. A blend of GS/UHMWPE (GUR 1020, Celanese, Grover, N.C., USA) was prepared using the sieved, then ground GS. The GS powder was mixed with UHMWPE powder and the blend was further mixed using a mechanical mixer (Glen Mills Turbula T2F Mixer, NJ, USA) operated at 49 rpm for 30 minutes. Blends with different concentrations were prepared using this method: 4.22 wt. % GS in UHMWPE, and 8 wt. % GS in UHMWPE were weighed and placed in the mechanical mixer and blended at 49 rpm for 30 minutes.

### Example 2. Compression Molding of GS/UHMWPE Blend

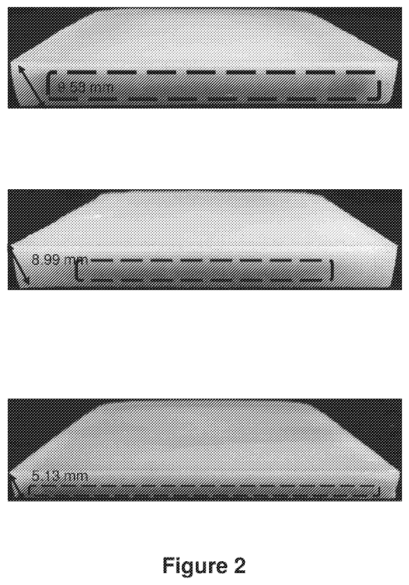

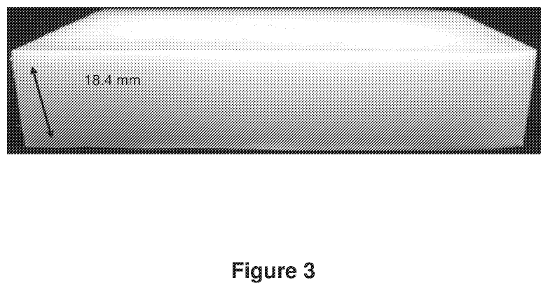

GS/UHMWPE with 4.22 wt. % GS was prepared using the method described in Example 1 and the blend was poured in the cavity of a mold pre-heated to approximately 180° C. The mold used here was made from aluminum bronze. The plunger was also preheated (180° C.) and was placed inside the mold cavity. The plunger mold assembly was placed between platens (heated to 170° C.) of a molding press (Carver Auto-Series Model 3895 Press, 30 ton capacity, Carver Inc., IN, USA) and was loaded to consolidate the blend at temperature (in this case 180° C.) for 6 min. Three samples were molded using this method under different loads to achieve a consolidation pressure of 10 MPa, 20 MPa or 40 MPa inside the mold cavity. The molded pieces were sectioned, and the extent of discoloration was visually assessed. The cross-section of the samples showed varying degrees of discoloration from pale yellow to dark brown (FIG. 2). All three samples had non-uniform discoloration throughout the bulk.

### Example 3. Wet Blending of GS/UHMWPE Blend

0.6 g of GS was dissolved in 12 ml of deionized water in a beaker. 13.4 g of UHMWPE was wetted in 12 ml of acetone in another beaker. Aqueous GS solution was mixed with the acetone-wetted UHMWPE powder by pouring the GS solution over UHMWPE slowly while the solution was vigorously stirred with a mechanical mixer. The resulting mixture was kept stirring on a heating plate at 45° C. until the mixture was visibly dry. The blend than placed in a vacuum oven at 45° C. to dehydrate the blend for about 16 hours. The dehydrated blend was further mixed with a planetary mixer for 30 min. at 49 rpm. The mixed blend was further dried by another vacuum oven treatment.

### Example 4. Forming a Pellet of GS/UHMWPE Blend

GS/UHMWPE blends with 4.22 and 8 wt. % GS were prepared as described in Example 1. Briefly GS was sieved through 75 pm sieve and was mixed with UHMWPE in a mechanical mixer. Each blend was pelletized: The blends, 14 grams of each were separately poured into the cavity of a rectangular mold (inner dimensions of 85 mm length by 50 mm width) and the plunger was placed in the mold cavity. The mold used here was made from aluminum bronze. The mold/plunger assembly was placed in a compression molding press (Carver Auto-Series Model 3895 Press, 30 ton capacity, Carver Inc., IN, USA) and was loaded at room temperature (10° C.-40° C.) at a pressure of 20 MPa for 15 minutes. Then, the mold was unloaded and the pellet was removed from the mold.

### Example 5. Vacuum Treatment of the GS/UHMWPE Pellet

The pellets of GS/UHMWPE that were prepared as described in Example 4 were placed in a vacuum oven (Thermo Scientific Lindberg/Blue M, Thermo Scientific, Waltham, Mass., USA) supported by the rim of a glass petri dish such that most of the surfaces of the pellets were not in contact with a solid surface. The oven was heated to 45° C. and maintained in vacuum (<1 inch Hg) for about 18 hours at 45° C. Subsequently, the oven chamber was backfilled with argon gas at the end of the vacuum treatment period to reach atmospheric pressure and the sample was removed.

### Example 6. Compression Molding of the Vacuum Treated, Pelletized GS/UHMWPE Blend

GS/UHMWPE blend containing 4.22 wt. % GS with varying thicknesses were prepared as described in Example 1. Pellets made with GS/UHMWPE blend containing 4.22 wt. % GS were prepared as described in Example 4. The pellets were vacuum dehydrated as described in Example 5. The pellets were consolidated: the mold and the plunger were first heated for 45 min in an air convection oven at 180° C. The vacuum-treated pellet was placed inside the mold cavity of the preheated mold and the preheated plunger was placed inside the mold cavity. A compression molding press was used to consolidate the vacuum-treated pellets. The platens were first heated to 170° C. and then the plunger mold assembly was placed between the platens. The mold was loaded to reach a pressure of 20 MPa to consolidate the pellet. Longer molding duration was used to consolidate thicker pellets (Table 2). The mold and plunger assembly were cooled under load and the consolidated blend was removed from the mold.

The consolidated samples were cut in the direction of compression (molding) to observe the uniformity of color change within the bulk. As seen in FIG. 6, the cross-section showed a uniform color throughout the bulk of the sample. In comparison to the samples from Example 2, which were consolidated from powder form of the blends, the consolidation of the vacuum-treated pellets resulted in less color change and any discernible color was uniform with the latter. FIG. 2 shows a gentamicin sulfate blend of UHMWPE polymeric material prepared as described above by blending, pelletizing, vacuum-treatment and compression molding.

### Example 7. XPS Data of Powder Molding Vs Pellet Molding

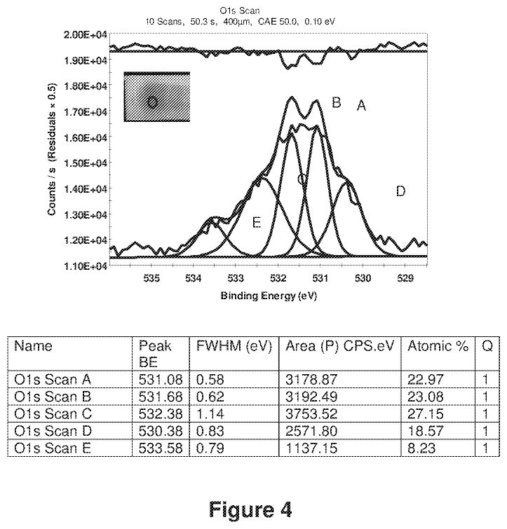

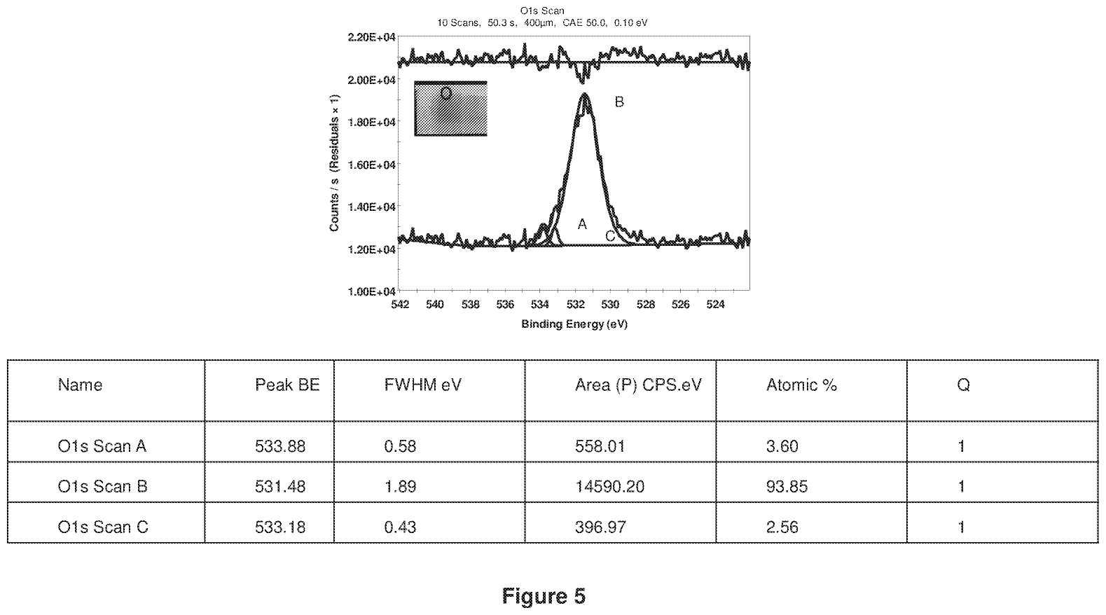

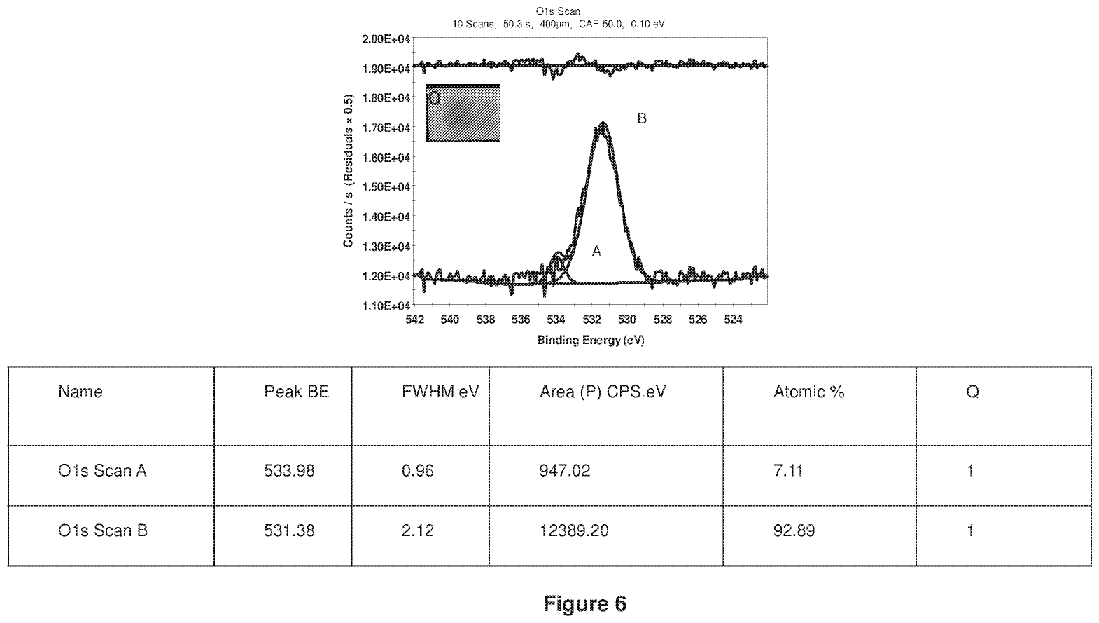

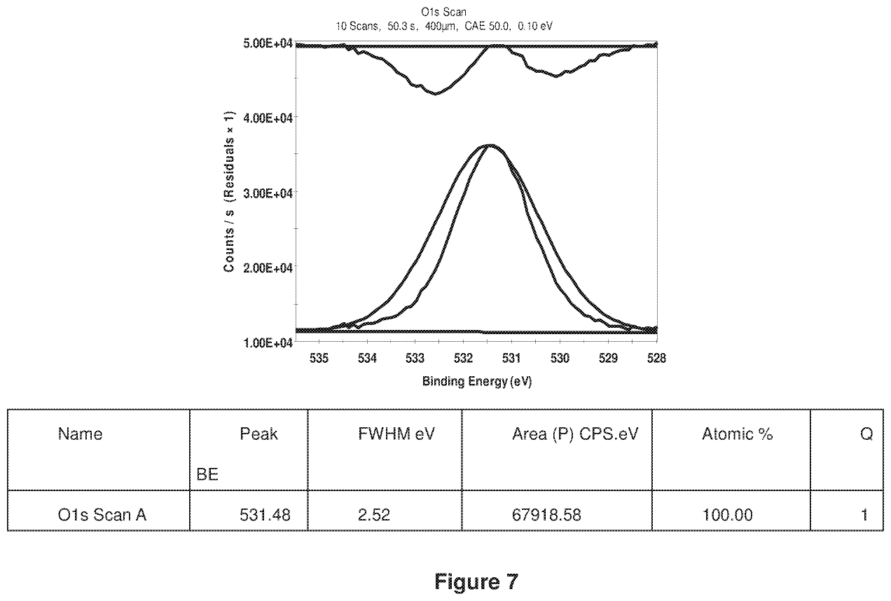

Two GS/UHMWPE blends with 4.22 wt % GS which was prepared as described in Example 1 were molded as described in Example 2 (powder-molded sample) and Example 6 (pellet-molded sample). Subsequently, the molded blocks were microtomed to 100 pm thin sections by using a sliding microtome (Leica SM2400, Leica Biosystems Inc., IL, USA). The microtomed GS/UHMWPE films were analyzed using an XPS (K-Alpha+, Thermo Fisher Scientific, Waltham, Mass., USA). O1s data was collected from different spots with visibly discolored center, less discolored skin and the intersection between the skin and the center of the powder-molded sample. For the pellet molded sample, O1s data was collected from 3 different regions, specifically the skin, center and interface between the two as shown in the photographs in FIG. 4, FIG. 5 and FIG. 6. Spectra collected from the powder-molded sample showed multiple states of oxygen and these states varied from the center to skin (FIG. 4, FIG. 5, FIG. 6). In contrast, the spectra collected from the pellet-molded sample as in Example 6 showed no change in the state of oxygen throughout the sample depth (FIG. 7, FIG. 8, FIG. 9). These results suggested that pelletizing the GS/UHMWPE blend, vacuum treating the pellet and subsequently consolidating the pellet resulted in a uniform sample.

### Example 8. Surface Layered Compression Molding

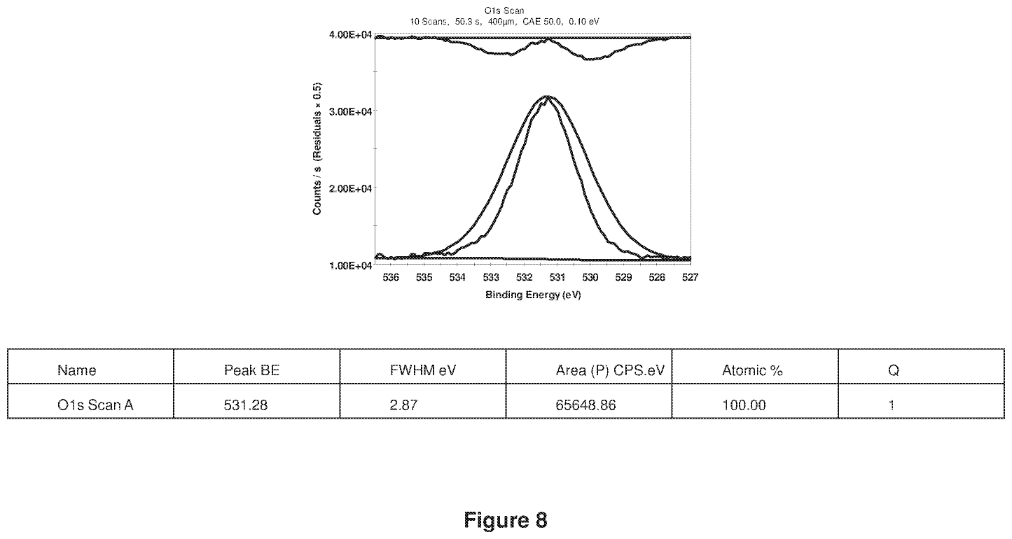

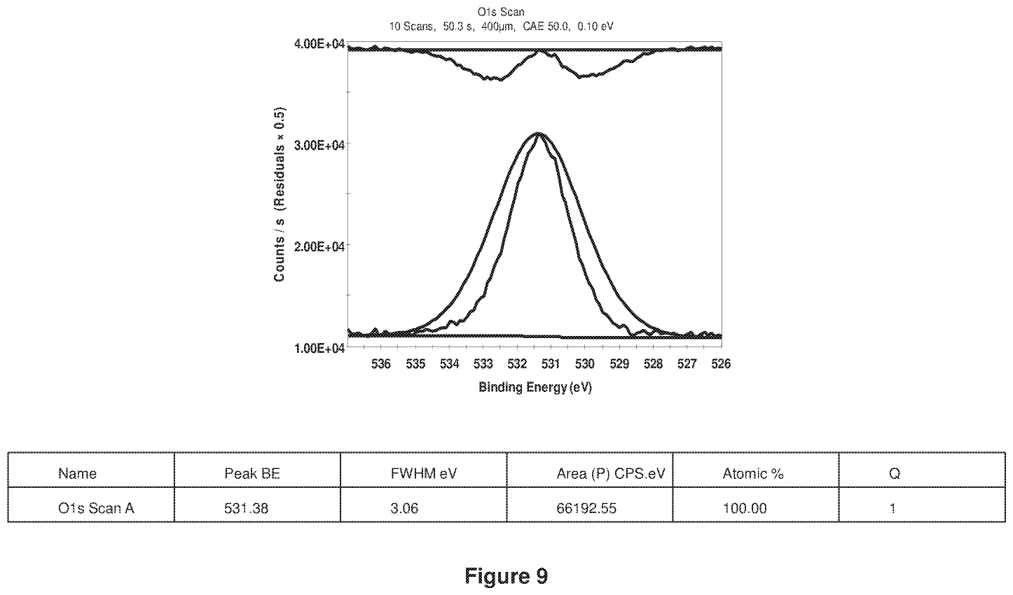

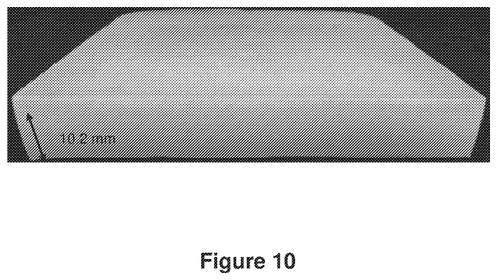

Virgin UHMWPE powder (42 grams) was poured into the cavity of a rectangular mold (dimensions, aluminum bronze), the plunger was placed into the mold and the mold was placed in between the platens of a compression molding press. The press was loaded for 15 minutes at room temperature such that the peak pelletization pressure was 20 MPa. After 15 minutes, the load was removed, the plunger was separated from the mold, and a GS/UHMWPE blend (9 grams; prepared as described in Example 4) was layered on top of the pelletized virgin UHMWPE. The plunger was replaced inside the mold cavity, the mold assembly was placed in between the platens of a compression molding press where the top platen was preheated to 155° C. (and the bottom platen was not actively heated) and loaded for 4 minutes with a load set point such that the peak molding pressure was 20 MPa. Finally, the mold was cooled down to 25° C. by circulating house water (15-20° C.) in the platens of the press. The thin GS layer was discolored while the rest of the block was not visibly discolored (FIG. 10).

### Example 9. GS Calibration and GS Quantification in LC/MS

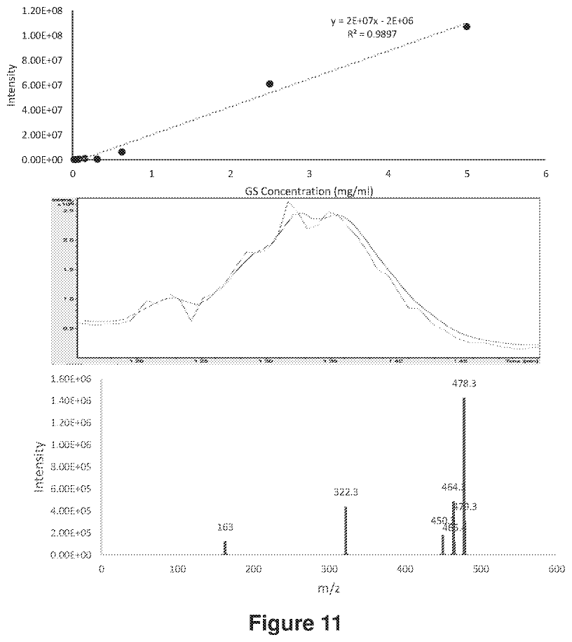

Nine gentamicin sulfate (GS) solutions in phosphate buffered saline (PBS) were prepared with concentrations of 10, 5, 2.5, 1.25, 0.63, 0.31, 0.16, 0.078, 0.04, 0.02 mg/ml and intensity of GS isomers peaks were analyzed with LC/MS. The calibration samples were analyzed in an Agilent LC/MS instrument (Agilent 1200 series Liquid Chromatography, 6310 Mass Spectrometry, CA, USA) with electrospray ionization. Measurements were performed with C8 (Zorbax C8, 2.1 mm, 150 mm, Agilent) and C18 (Acclaim 120 C 18, 2.1 mm, 100 mm, ThermoFisher) columns separately with a mobile phase of acetonitrile and deionized water with 0.1 wt. % formic acid. The flow rate for injection was adjusted to 0.200 ml/min and mobile phase used was a gradient mixture of water (95%) and acetonitrile (5%). All peaks that were present in the GS total ion chromatogram were integrated to obtain the corresponding mass spectrum of elution aliquots (FIG. 11B). A calibration curve was generated by plotting the cumulative integration of all peaks versus GS concentration (FIG. 11A). GS has three main isomers with m/z ratios that appear in between 447-451, 463-466 and 477-481. We used these three peaks for the integration used in the calibration (FIG. 11C).

### Example 10. GS Elution Experiment

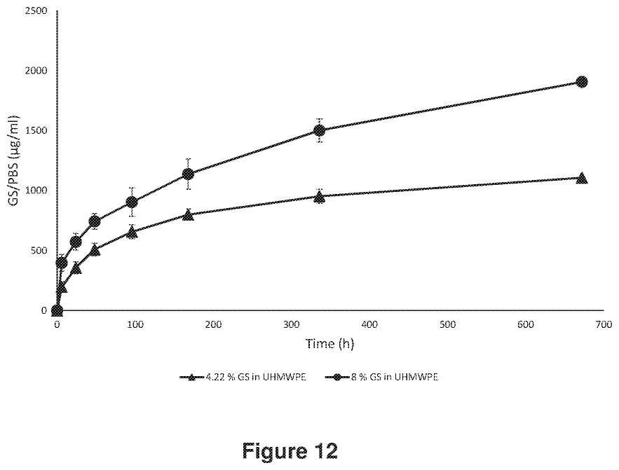

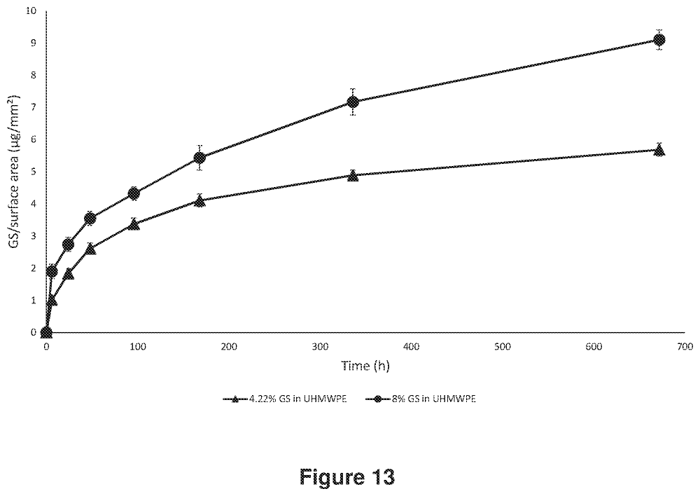

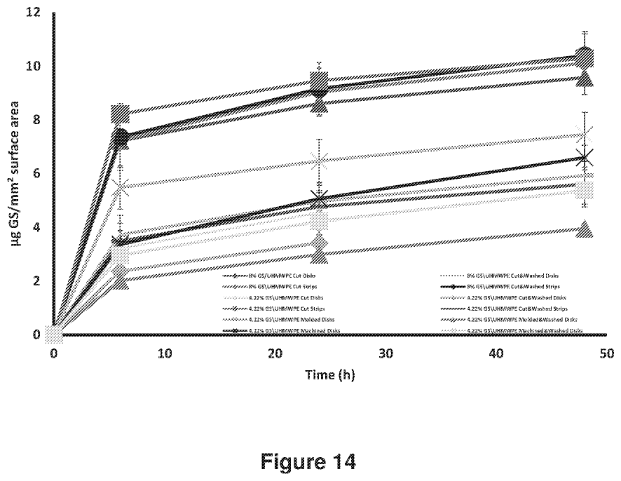

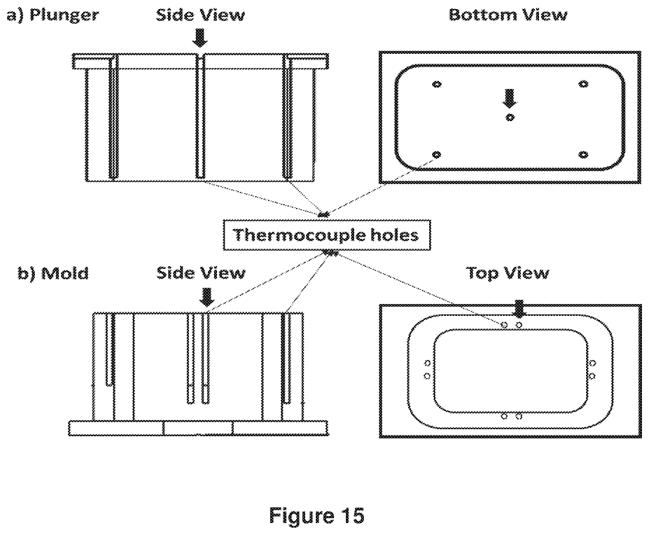

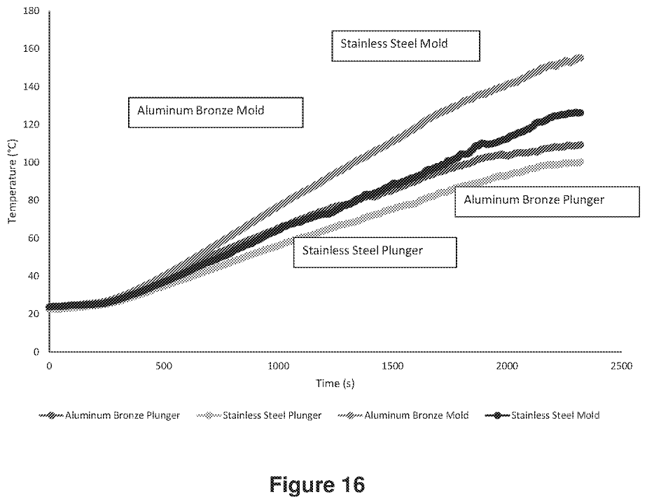

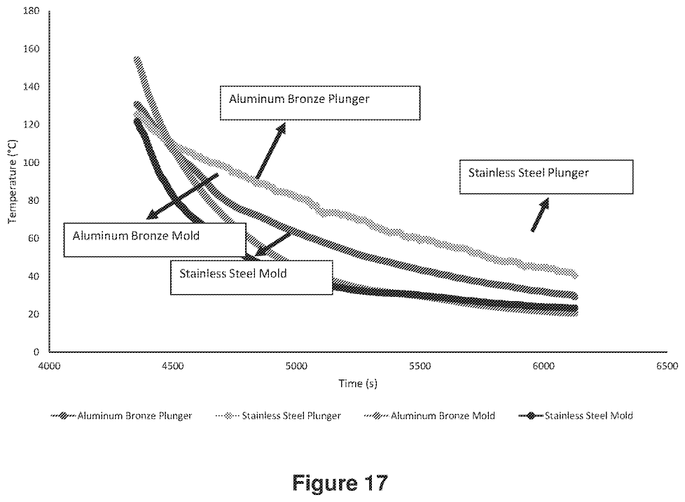

Blends of GS/UHMWPE with 4.22 wt. % GS and 8 wt. % GS were pelletized, vacuum treated and molded as described in Example 6 above and were cut into strips (20×5×3 mm). The strips (n=3) were placed in sterile polypropylene syringes (5 mL) in 1.5 ml of PBS 7.4 (Gibco 1X). Syringes were kept upright in an incubator shaker (222DS Benchtop Shaking Incubator, Labnet International, NJ, USA) at 37° C., and were agitated at 100 rpm. The PBS in the syringes were collected at 6 h, 24 h, 48 h, 96, 168 h, 336 h, 672 h and the PBS was replenished at each time point. Entire PBS content in the syringe was collected for LC/MS analysis before replenishing the PBS in the syringe and continuing the elution experiment. The collected PBS was transferred to 2 mL screw amber autosampler vials (Agilent Technologies, USA) and the vials were closed with caps and septa. The collected PBS samples were analyzed by LC/MS (Agilent 1200 series Liquid Chromatography, 6310 Mass Spectrometry, CA, USA). The vials were placed in the autosampler tray and the measurements were performed with C18 (Acclaim 120 C 18, 2.1 mm, 100 mm, ThermoFisher) column with a mobile phase of deionized water and acetonitrile with 0.1 wt. % formic acid. The flow rate was 0.2 ml/min and 10 μl samples from the vials were injected for the measurements. GO chromatograms were analyzed as described in Example 9 to obtain GS concentration measurements. The total mass of GS found in each syringe at each incremental time point was calculated using the concentration measurements from LC/MS analysis. The total GS amount was normalized to the total surface area of each strip in each syringe and reported as ng of GS per mm² of strip. The GS elution was higher with the higher GS concentration in UHMWPE (FIG. 12, FIG. 13, FIG. 14).

### Example 11. Effect of Manufacturing Method on Elution of GS

Blends of GS/UHMWPE with 4.22 wt. % GS and 8 wt. % GS were pelletized, vacuum treated and molded to 3 mm-thick blocks as described in Example 6. For GS/UHMWPE with 4.22 wt. % GS, four sets of samples each with 6 replicas were prepared from the molded samples: (i) 20×5×3 mm strips cut with a razor blade, (ii) disks (4.5 mm radius and 3 mm thickness) cut with a biopsy punch, (iii) disks (4.5 mm radius and 3 mm thickness) direct compression molded with a pin mold; (iv) disks (4.5 mm radius and 3 mm thickness) machined with a ShopBot Desktop CNC using a 0.125 inch thick bid at 200 rpm. For GS/UHMWPE with 8 wt. % GS, two sets of samples each with six replicas were prepared from the molded samples: (i) 20×5×3 mm strips cut with a razor blade and (ii) disks (4.5 mm radius and 3 mm thickness) cut with a biopsy punch.

Half of every set of samples (n=3) was washed in a 50 ml Falcon tube with PBS for 30 seconds by shaking. Strips were then placed in sterile syringes in 1.5 ml of PBS and disks were placed in sterile syringes in 2.0 ml of PBS. All syringes were placed in an incubator shaker at 37° C., at 100 rpm for GS elution to take place. Elution medium was collected after 6, 24 hours and 48 hours by transferring to a HPLC vial. Subsequently, empty syringes were filled with fresh PBS. The amount of GS eluted in to each elution medium were measured as described in Example 10. Manufacturing technique mostly affected the burst release of GS (FIG. 14).

### Example 12. Tensile Testing

Tensile testing was performed with dogbone-shaped samples that were stamped from consolidated 3 mm thick samples of GS/UHMWPE containing 4.22 and 8 wt. % GS as described in Example 2 and Example 6 in accordance with ASTM D638. Dogbones were stamped from molded thin sections by using a type V tensile stamp (Dewes-Gumbs Die Company, NY, USA). The dogbones were tested in tension at a crosshead speed of 10 mm/min by using an MTS tensile tester (MTS Insight 2, MTS Systems Inc.). True strain and elongation was measured by a laser extensometer. The calculated mechanical values are listed in Table 2.

TABLE 2
Ultimate tensile strength and elongation at break results of
4.22 wt. % and 8 wt. % GPE blocks

| | UTS (Mpa) | EAB (wt. %) |
|---|---|---|
| GS/UHMWPE with 4.22 wt | 38 ± 2 | 327 ± 6 |
| % (Example 2) | | |
| GS/UHMWPE with 4.22 | 37 ± 1 | 372 ± 7 |
| wt. | | |
| % (Example 6) | | |
| GS/UHMWPE with 8 wt. % | 36 ± 1 | 385 ± 28 |
| (Example 6) | | |

### Example 13. Comparing Temperature Profiles of the Stainless Steel and Aluminum Bronze Type Molds

We measured the time-temperature history inside the mold and the plunger during a molding cycle with both stainless steel or aluminum bronze (Types C95400) used in the fabrication of the mold/plunger assembly. We instrumented the mold and the plunger with thermocouples (5SRTC-GG-T-20-120, OMEGA Engineering, INC., Norwalk, Conn., USA). The thermocouples were in the holes shown with arrows on FIG. 13-1, one in the plunger and other in the mold. The mold and the plunger were assembled and were placed between the platens of the molding press at room temperature. The molding press was set to 170° C. for both top and bottom platens. The platens were closed to touch to the surfaces of the plunger and the mold and heating was started. Once the temperature stabilized, the platens were cooled to about room temperature by circulating cold water (15-20° C.). Aluminum bronze mold/plunger heated up and cooled down more rapidly than the stainless steel. It may be beneficial to use high thermal conductivity materials, such as aluminum or aluminum bronze, as mold and plunger materials to minimize the heating and cooling during consolidation of polymeric material blends to minimize exposure time to elevated temperatures.

### Example 14. Microwave Treatment of GS/UHMWPE Blends

A 3 mm thick GS/UHMWPE pellet with 4.22% wt. GS was prepared as described in Example 4. The pellet than was placed in a microwave oven (1.2 CU. FT. 1100 W Touch, Emerson Radio Corp, NJ, USA) supported by the rim of a jar such that its surfaces were mostly free from direct contact with a solid surface. The pellet was subjected to microwave heating for 3 minutes at a power setting of 1100 W while rotating on the turntable. Separately, a GS/UHMWPE blend (in powder form) with 4.22% wt. GS was prepared as described in Example 1. The powder blend was placed in the microwave oven in a polyethylene jar on the turntable of the microwave oven. Two blends of the same formulation (4.22% GS in UHMWPE) were treated in the microwave oven, one for 5 min. and the other for 10 min. both at a power setting of 1100 W while rotating on the turntable.

### Example 15. Compression Molding of the Microwave Treated, Powder GS/UHMWPE Blend

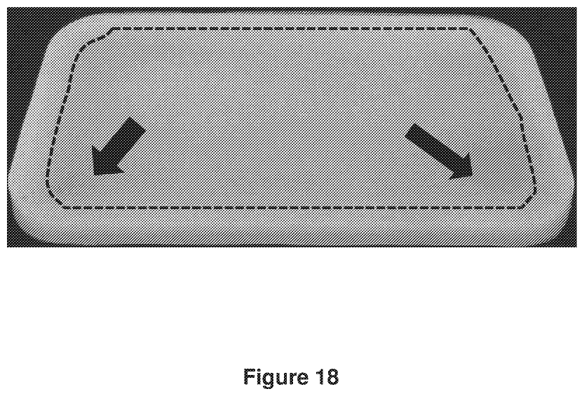

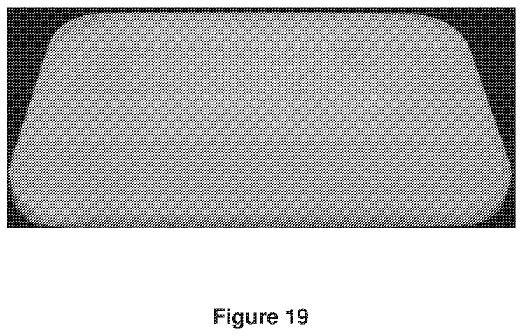

The microwave-treated powder GS/UHMWPE blends prepared as described in Example 14 were consolidated using a rectangular aluminum-bronze mold and plunger. The mold and plunger were first heated for 45 min in an air convection oven that was preheated to 180° C. The two microwave-treated (5 min and 10 min) GS/UHMWPE powder blend samples were consolidated separately. Consolidation consisted of placing the powder blend in the heated mold cavity then pressurizing the mold cavity with the heated plunger in compression molding press. The platens of the press were first heated to 170° C. and then the plunger mold assembly with GS/UHMWPE powder in it was placed between the compression molding platens. The mold was loaded to reach a pressure of 20 MPa inside the mold cavity and held at that pressure for 8 minutes. The resulting molded piece had a thickness of 3 mm. The mold plunger assembly was cooled under load and the molded blend was removed (FIG. 18, FIG. 19). There was substantially more discoloration with the 5-min microwave-treated block then the one that was heated in the microwave for 10 min. Longer duration exposures to microwave oven may be preferable for certain additives to minimize discoloration during consolidation.

### Example 16. Compression Molding of the Microwave-Treated, Pelletized GS/UHMWPE Blend

The microwave-treated GS/UHMWPE pellets prepared as described in Example 14 were consolidated using a rectangular aluminum-bronze mold and plunger. The mold and plunger were first heated for 45 min in an air convection oven that was preheated to 180° C. The microwave-treated GS/UHMWPE pellet was placed inside the mold cavity (85×50×55 mm) of the pre-heated mold and the pre-heated plunger was placed inside the mold cavity. A compression molding press was used to consolidate the microwave-treated pellets. The platens of the press were first heated to 170° C. and then the plunger mold assembly was placed between the compression molding platens. The mold was loaded to reach a pressure of 20 MPa inside the mold cavity and maintained at pressure and temperature for 8 minutes. The molded piece had a thickness of 3 mm. The mold plunger assembly was cooled under load and the molded blend was removed from the mold.

### Example 17. Lyophilization of GS/UHMWPE Blends

The GS\UHMWPE powder blends are prepared as described in Example 1 and Example 4 with 4.22% wt. Gentamicin Sulfate. The powder blends are placed on the shelf of the lyophilizer and cooled down to 0° C., −10° C., −20° C., −30° C., −40° C., −50° C., −60° C., −70° C., −80° C., −90° C., −100° C., −150° C., −196° C. or any temperature in between and kept at the desired temperature for a duration of 10 min, 20 min, 30 min, 40 min, 50 min, 1 h, 2 h, 3 h, 4 h, 5 h, 6 h, 7 h, 8 h, 9 h, 10 h, 15 h, 24 h or less than 10 min or more than 24 h or anywhere in between. The pressure in the lyophilizer can be reduced with the use of vacuum pump to 6 mbar, 5 mbar, 4 mbar, 3 mbar, 2 mbar, 1 mbar, 0.5 mbar, 0.4 mbar, 0.3 mbar, 0.2 mbar, 0.1 mbar, 0.05 mbar, 0.04 mbar, 0.02 mbar, 0.01 mbar, 0.005 mbar, 0.004 mbar, 0.002 mbar, 0.001 mbar or in any pressure in between. Alternatively, freeze-drying can be continued but increasing the temperature in the chamber to 10° C. or lower, 20° C., 30° C., 40° C., 50° C. or below 10° C. or above 50° C. or any temperature in between for a duration of 10 min or shorter, 20 min, 30 min, 40 min, 50 min, 1 h, 2 h, 3 h, 4 h, 5 h, 6 h, 7 h, 8 h, 9 h, 10 h, 15 h, 24 h or less than 10 min or more than 24 h or anywhere in between.

### Example 18. Dry Blending and Compression Molding of GS and Sulfite Additives with UHMWPE

4.22% and 8% GS can be blended with UHMWPE as described in Example 1. Subsequently, sodium sulfite or sodium bisulfite or sodium metabisulfite or potassium metabisulfite or calcium sulfite or calcium hydrogen sulfite or potassium hydrogen sulfite can be blended with the respective GS/UHMWPE powder blends. The amount of sulfite additive can be 0.5%, 1%, 2%, 3%, 4%, 4.22%, 5%, 6%, 7%, 8% by weight or less than 0.5% or more than 8% or any weight percent in between. The GS and sulfite blended UHMWPE can be molded as described in Example 2.

### Example 19. Wet Blending and Compression Molding of GS and Sulfite Additives with UHMWPE

GS and sodium sulfite or sodium bisulfite or sodium metabisulfite or potassium metabisulfite or calcium sulfite or calcium hydrogen sulfite or potassium hydrogen sulfite are dissolved in deionized water. The solution can be then poured on UHMWPE powder in a beaker and stirred vigorously by using a stir bar and a magnetic stirrer. Subsequently the wet blend can be dried using methods such as heating the blend in air or in inert gas, subjecting the blend to vacuum either at room temperature and/or at elevated temperature and/or by using other methods. The amount of sulfite additive can be 0.5%, 1%, 2%, 3%, 4%, 4.22%, 5%, 6%, 7%, 8% by weight or less than 0.5% or more than 8% or any weight percent in between. The GS and sulfite blended UHMWPE can be molded as described in Example 2.

### Example 20. Dry Blending and Compression Molding of GS and Pyridoxine Hydrochloride (Vitamin B6) Additives with UHMWPE

4.22% and 8% GS can be blended with UHMWPE as described in Example 1. Subsequently, Pyridoxine hydrochloride (vitamin B6) can be blended with GS/UHMWPE. The amount of vitamin B6 can be 0.5%, 1%, 2%, 3%, 4%, 4.22%, 5%, 6%, 7%, 8% by weight or less than 0.5% or more than 8% or any weight percent in between. The GS and B6 blended UHMWPE can be molded as described in Example 2.

### Example 21. Wet Blending and Compression Molding of GS and Sulfite Additives with UHMWPE

GS and Pyridoxine hydrochloride (vitamin B6) are dissolved in deionized water. The solution can be then poured on UHMWPE powder in a beaker and stirred vigorously by using a stir bar and a magnetic stirrer. Subsequently the wet blend can be dried using methods such as heating the blend in air or in inert gas, subjecting the blend to vacuum either at room temperature and/or at elevated temperature and/or by using other methods. The amount of vitamin B6 can be 0.5%, 1%, 2%, 3%, 4%, 4.22%, 5%, 6%, 7%, 8% by weight or less than 0.5% or more than 8% or any weight percent in between. The GS and vitamin B6 blended UHMWPE can be molded as described in Example 2.

It is to be understood that the description, specific examples and data, while indicating exemplary embodiments, are given by way of illustration and are not intended to limit the present invention. Various changes and modifications within the present invention, including combining embodiments in whole and in part, will become apparent to the skilled artisan from the discussion, disclosure and data contained herein, and thus are considered part of the invention.

1. A method of making a consolidated blend of polymeric material with a therapeutic agent comprising:
a. providing a polymeric material,
b. blending the polymeric material with one or more therapeutic agent(s) at least one of which is an antibiotic,
c. pelletizing the blended polymeric material,
d. environmentally treating pelletized polymeric material,
e. completely consolidating the environmentally treated pellet.

2. A method of making a consolidated blend of polymeric material with a therapeutic agent comprising:
a. providing a polymeric material,
b. blending the polymeric material with one or more therapeutic agent(s),
c. pelletizing the blended polymeric material,
d. environmentally treating pelletized polymeric material,
e. completely consolidating the environmentally treated pellet.

3. A method of making a medical implant comprising:
a. providing a polymeric material,
b. blending the polymeric material with one or more therapeutic agents at least one of which is an antibiotic,
c. pelletizing the blended polymeric material,
d. environmentally treating pelletized polymeric material,
e. completely consolidating the environmentally treated pellet.

4. A medical implant made by the method comprising:
a. providing a polymeric material,
b. blending the polymeric material with one or more therapeutic agents at least one of which is an antibiotic,
c. pelletizing the blended polymeric material,
d. environmentally treating pelletized polymeric material,
e. completely consolidating the environmentally treated pellet.

5. A method of making a consolidated blend of polymeric material comprising:
a. providing a polymeric material,
b. blending the polymeric material with therapeutic agents at least one of which is an antibiotic,
c. pelletizing the blended polymeric material,
d. microwave treatment of pelletized polymeric material,
e. completely consolidating the environmentally treated pellet.

6. A method of making a medical implant comprising:
a. providing a polymeric material,
b. blending the polymeric material with one or more therapeutic agents at least one of which is an antibiotic,
c. pelletizing the blended polymeric material,
d. microwave treatment of pelletized polymeric material,
e. completely consolidating the environmentally treated pellet.

7. A medical implant made by the method comprising:
a. a. providing a polymeric material,
b. blending the polymeric material with therapeutic agents at least one of which is an antibiotic,
c. pelletizing the blended polymeric material,
d. microwave treatment of pelletized polymeric material,
e. completely consolidating the environmentally treated.

8. A method of making a consolidated blend of polymeric material comprising:
a. providing a polymeric material,
b. blending the polymeric material with one or more therapeutic agents at least one of which is an antibiotic,
c. pelletizing the blended polymeric material, thereby forming a pellet,
d. microwave treating the pellet,
e. environmentally treating the pellet,
f. completely consolidating the microwaved and environmentally treated pellet.
